(19) United States
(12) Patent Application Publication
(10) Pub. No.: US 2014/0127307 A1
(43) Pub. Date: May 8, 2014

(54) MICROPELLET COMPOSITIONS COMPRISING PANCREATIN CONTAINING DIGESTIVE ENZYME MIXTURE

(71) Applicant: APTALIS PHARMA LIMITED, Wicklow (IE)

(72) Inventors: Gopi M. VENKATESH, Vandalia, OH (US); Craig Kramer, New Labanon, OH (US); Flavio Fabiani, Merate (IT); Luigi Mapelli, Milano (IT); Giovanni Ortenzi, Monza (IT); Massimo Latino, Milan (IT)

(73) Assignee: APTALIS PHARMA LIMITED, Wicklow (IE)

(21) Appl. No.: 13/667,859

(22) Filed: Nov. 2, 2012

(51) Int. Cl. A61K 9/16; A61K 38/47; A61K 38/48; A61K 38/54; A61K 38/46

(52) U.S. Cl. CPC A61K 9/1676; A61K 38/54; A61K 38/465; A61K 38/48; A61K 38/47
USPC 424/491; 424/94.21; 424/94.6; 424/94.61; 424/94.63; 424/94.64; 435/196; 435/198; 435/201; 435/207; 435/209; 435/212; 435/213

(57) ABSTRACT

The present invention relates to a small particle size composition comprising pancreatin containing digestive enzymes for use in patients in need, including pediatric, geriatric, and adult patients, particularly those patients with dysphagia or wherein enteral administration using such composition would be suitable. In addition, the invention is directed to the composition as particles, such as micropellets or microgranules having a high potency, high useable yield and at least 10%-90% of 400-800 μm. Furthermore, the composition optionally has an improved enteric coating and concomitant improved stability and enzyme activity compared to conventional prepared enterically coated pancreatic enzyme particles.

### CROSS REFERENCE TO RELATED APPLICATIONS

The present application claims priority to U.S. Provisional Application No. 61/330,768 filed May 3, 2010, which is incorporated herein by reference in its entirety for all purposes.

### BACKGROUND OF THE INVENTION

Pancreatic enzyme supplements were available in the United States before the passage of the 1938 Food, Drug and Cosmetic Act. Pancreatin enzymes with and without enteric coating were formulated and marketed until recently without the regulatory approval.

Exocrine pancreatic insufficiency (EPI) caused by various diseases affecting the pancreas, such as pancreatitis, pancreatectomy, cystic fibrosis (CF), etc, has been a condition for which pancreatic enzyme supplements have been used. Pancrelipase and other pancreatic enzymes products (PEPs) can be administered to at least partially remedy the enzyme deficiency in the production and/or secretion of pancreatic enzymes that are necessary to digest nutrients in food. Without these supplements, patients become severely nutritionally impaired. This nutritional impairment can be life threatening if left untreated, particularly in the case of infants.

Physiologically acceptable pancreatic enzyme mixtures with lipolytic, proteolytic and amylolytic activity of microbial origin and/or especially digestive enzyme mixtures of animal origin, catalyze the hydrolysis of fats into glycerol and fatty acids, starch into dextrin and sugars, and proteins into amino acids and derived substances. The pancreatic enzymes are not absorbed from the gastrointestinal (GI) tract in any appreciable amount. The primary efficacy endpoint relative to the pancreatic enzyme mixtures is the mean difference in the coefficient of fat absorption (CFA) between pancreatic and placebo treatment. The CFA is determined by a 72-hour stool collection during the treatment, when both fat excretion and fat ingestion are measured. Each patient's CFA during placebo treatment is used as the no-treatment CFA value.

Pancrelipase is mainly a combination of three enzyme classes: lipase, protease and amylase, together with their various co-factors and coenzymes, which is used to supplement loss of or low levels of digestive enzymes used in a number of essential body processes. These enzymes are produced naturally in the pancreas and are important in the digestion of fats, proteins and carbohydrates. Pancrelipase is typically prepared from porcine pancreatic glands, although other sources can also be used, for example those described in U.S. Pat. No. 6,051,220; U.S. 2004/0057944; 2001/0046493; and WO 2006044529. The enzymes catalyze the hydrolysis of fats into glycerol and fatty acids, starch into dextrin and sugars, and protein into amino acids and derived substances.

Pancreatin is a digestive enzyme that is also used to supplement loss of or low levels of digestive enzymes used in a number of essential body processes. Pancreatin comprises the pancreatic enzymes: lipase, amylase, and protease. Pancreatin differs from pancrelipase for the activity levels of the three major enzymes.

Digestion by pancreatic enzymes and absorption of the metabolites can take place throughout GI transit. However, pancreatic enzymes show optimal activity under near neutral to slightly alkaline conditions. Under gastric conditions, i.e., in the presence of acid and pepsin, most of the enzymes, especially the lipases, may be irreversibly inactivated with a resulting loss in biological activity. Therefore, exogenously administered enzymes are generally protected against gastric inactivation and remain intact during their transit through the stomach and into the duodenum. The enzymes are preferentially released in the duodenum within 5-30 minutes where pH≧5.5, as the activity of pancreatic enzymes is retained under such conditions and absorption of the metabolites takes place primarily in the upper segment of the intestine.

The protection of pancreatic enzymes has typically been effected by coating with an enteric coating polymer, which protects the enzyme composition against the acidic environment of the stomach and then provides release of the enzyme in the small intestine. Lipases are the most sensitive pancreatic enzymes and are the most important single enzymes in the treatment of malabsorption. Lipase activity is typically monitored to determine the stability of an enzyme composition containing lipase. Although the protection is desired, uncoated preparations are also found in commerce.

Pancreatic enzyme compositions are typically coated using conventional pan coating or fluid bed coating methods in which pancreatic enzyme particles (e.g. granules, microtablets, minitablets, etc.) are coated with a polymer solution or polymer suspension at temperatures sufficiently high so as to rapidly evaporate the coating solvent, and thereby provide enzyme particles with a relatively dry, solidified polymeric coating. However, it is difficult to provide uniform polymer coatings using conventional pan coating or fluid bed coating methods on pancreatic enzyme particles, such as those suitable for infant feeding via sprinkling. Furthermore, such coating conditions subject the pancreatic enzyme particles to heat, moisture, and oxygen which can affect the enzyme stability, and thus lead to loss of enzyme activity. Therefore, it would be desirable to obtain coated pancreatic enzyme particles under less stringent conditions which maintain enzyme activity and stability.

Conventional pancreatic enzyme compositions are also comprised of relatively large pancreatic enzyme particles, which are not well suited for pediatric patients, or for enteral administration, such as nasogastric (NG), nasojejunal (NJ), percutaneous endoscopic gastrostomy (PEG) or jejunostomy tubes. T. Sipos in U.S. Pat. No. 4,079,125 and U.S. Pat. No. 5,302,400 teaches the preparation of pancreatin micropellet cores which are subsequently coated with an enteric polymer comprising granulating a pancreatin enzyme mixture, a binder, a disintegrant, an effervescent couple, and bile acids, extruding and spheronization. The enteric coated pellets had a particle diameter of 1.4-2.0 mm. U.S. Pat. No. 4,280,971 teaches the preparation of 1.5-1.7 mm long strands by extruding a mixture of pancreatin, magnesium stearate granulated with isopropanol and spheronizing the dried strands by alternately spraying an isopropanol solution of povidone or polyethylene glycol and pancreatin. The enteric coated pellets had a particle size of 0.5 to 4 mm, preferably 1 to 2.5 mm. M. Maio discloses in US 20040101562 the preparation of microspheres comprising pancreatin enzymes and a hydrophilic low-melting polymer by melt granulation without using any solvents. The microspheres in the size range of 10 to 1500 μm are coated with an enteric polymer. US patents such as U.S. Pat. No. 5,378,462, U.S. Pat. No. 5,725,880, and publications such as US 20070148152 A1, US 20070148153 A1 teach the preparation of pancreatin micropellet cores which are subsequently coated with a gastroresistant polymer by extruding a mixture comprising pancreatin, polyethylene glycol (PEG 4000), and sufficient lower alcohol to achieve extrudable consistency and spheronizing the extrudate in the presence of liquid paraffin or isopropanol, the resulting pellets spheroidal to ellipsoidal in shape having a minimum diameter of 0.7-1.4 mm or larger. Difficulty in the formulating of the pancreatin compositions due to the instability of pancreatin in water and/or in the presence of moisture contributed to the aforesaid uses of the friendly solvents or low melting hydrophilic polymers suitable for the melt extrusion-spheronization. However, these melt extrusion methods typically produce pancreatin enzyme particles with wide particle size distributions. Given the aforesaid issues with the relatively large pancreatic enzyme particles of conventional pancreatic enzyme compositions and their method of production, a pancreatic enzyme composition comprised of relatively small enzyme particles with a narrow size distribution would be desirable.

Alternative pancreatic compositions have been made. K Y Weon et al. in WO 2007013752 disclose the preparation of pancreatin enzyme cores by spraying a dispersion comprising pancreatin enzymes in an aqueous solution of a polymer binder. The major drawback of this process is the difficulty in completing the pancreatin layering process within 60-90 minutes of the formulation preparation to avoid significant loss of activity. A. Margolin et al. in US 20010046493, US 20030017144, and WO 2006044529 disclose compositions comprising acid-stable microbial lipase crystals, microbial amorphous amylases, and microbial proteases, as well as methods for treating conditions including EPI without the need for gastroresistant coating. Galle et al. in US 20040057944 disclose the preparation of a microbial enzyme mixture which was reported to be suitable for treating EPI associated with CF. Although the preparation could retain biological activity in the pH range of 4 to 8, it was inactivated to some extent under in vivo conditions in CF patients with a stomach pH of 2.5-4. U.S. application Ser. No. 12/034A80 (Pub. No. US2008/0299185), describes stable digestive enzyme compositions. Scharpe discloses in U.S. Pat. No. 6,051,220 compositions comprising acid stable enzymes or complexes of microbial origin having maltase, amylase and dextrinizing activities containing up to 75% acid-stable lipase in combination with proteolytic and lipid splitting enzymes for use in the treatment of EPT. US 20080279839 (application Ser. No. 12/092,255) teaches the preparation of digestive enzyme compositions shown to be effective for treating EPI associated with CF, comprising at least one microbial derived lipase, at least one microbial derived amylase, and at least one protease preferably derived from plants, all of which are resistant to inactivation in the pH range of 2 to 8. U.S. Pat. No. 6,051,220, US 20010046493, US 2003017144, US 20040057944, WO 2006044529, and US 20080279839, also describe pancreatic enzyme preparations. U.S. Pat. No. 7,658,918 describes Zenpep®, a therapeutically effective doses of enzyme mixtures in the UPMC capsule form. Each capsule for oral administration contains enteric-coated beads (1.8-1.9 mm for 5,000 USP units of lipase, 2.2-2.5 mm for 10,000, 15,000 and 20,000 USP units of lipase).

Enzyme dosing should begin with 500 lipase units/kg of body weight per meal for those older than age 4 years to a maximum of 2,500 lipase units/kg of body weight per meal (or less than or equal to 10,000 lipase units/kg of body weight per day), or less than 4,000 lipase units/g fat ingested per day. A 500-milligram tablet of pancreatin usually has about 12,500 USP units of trypsin, 12,500 USP units of amylase and 1,000 USP units of lipase. Thus, patients are required to swallow several capsules at meal or snack, which makes adherence to dosing regimens and/or administration of currently available pancreatin preparations wherein 90% of the particles are >900 μm difficult.

Furthermore, due to significant differences in particle size distributions between pancreatin and food preparations, sychronization of movements of these two preparations during their transit through the gastrointestinal tract, although important from better absorption of fat and nutrients points of view, is often difficult to achieve, and is a major issue, especially in toddlers and infants. Choe et al. showed that gastric emptying was dependent only on size and not on chemical make-up of the pellets (Choe et al. in Euro J. Pharma Sci. 2001; 14,347-353), wherein 0.7 mm pellets containing APAP or caffeine, and gamma scintigraphy were used. They also showed that co-administration of 0.7 mm and 3.6 mm pellets (APAP) indicated that the smaller size 0.7 mm pellets (Caffeine) were emptied and absorbed sufficiently earlier than the larger size 3.6 mm (APAP) pellets with both the small Liquid Meal (by 35 min) and the Standard Meal (by 33 min) (P<0.05). The differences in the gastric emptying time for the pellets were not significant in the fasted state. J H Meyer et al. showed that 1 mm spheres emptied consistently (<<0.01, paired t-test) faster than 2.4 or 3.2 mm spheres when ingested together with 420 g or 100 g meals J H Meyer et al. Gastroenterology 1988; 94: 1315-1325). Based on the results of an open-label, randomized, single-dose, three-way crossover study 18 healthy male volunteers, 18-50 years of age with a washout period of at least 1 week between treatments (e.g., encapsulated enteric coated didanosine beads (1×200 mg; enteric beads of about 2 mm in diameter) and enteric coated didanosine mini-tablets (4×50 mg; enteric coated tablet of about 5 mm in diameter), B. Damle et al. concluded that the time of 1^{st} gastric emptying, defined as the time at which 95% or less of the radioactivity in the didanosine dosage form was observed in the stomach, for the enteric beads was higher than that obtained for the enteric tablet; however, the time that the 1^{st} quantifiable concentration of didanosine was observed in the plasma was lower for the enteric beads. Consequently, the lag time, defined as the time between gastric emptying and the onset of didanosine absorption, for the enteric beads was markedly lower than that for the enteric tablet (B. Damle et al. in Br. J. Clin. Pharmacol. 2002; 54(3): 255-261). Thus, there is an unmet need for an oral, delayed release pharmaceutically acceptable composition of pancreatin-containing digestive enzyme mixtures (e.g., typically protease, lipase, and amylase), with 90% of the enteric coated microparticles having a particle size of no more than 800 μm, for use in pediatric, geriatric, and adult patients. Furthermore, such preparations would enable easier administrations, especially in infants and toddlers.

It has now been found that pharmaceutically acceptable compositions comprising digestive enzyme mixture of pancreatin-containing micropellets with a narrow/small particle size distribution, high useable yield, and at least 10%-90% of the micropellets or microgranules are 400-800 μM in diameter, which are optionally coated with a gastroresistant coating or moisture-resistant coating, e.g., comprising a hydrophilic polymer and a plasticizer, optionally including a hydrophobic anti-tacking agent (such as talc, magnesium stearate, colloidal silicon dioxide and combinations thereof; further optionally a low viscosity ethylcellulose) for producing stable digestive enzyme compositions and dosage forms which are suitable for use in pediatric, geriatric, and adult patients to at least partially remedy the enzyme deficiency.

### BRIEF DESCRIPTION OF THE DRAWINGS

FIG. 1: photograph of Granurex GX-35-GXR processing insert with a conical rotating plate and VEC-Lab 3 with GXR-35 insert installed.

FIG. 2: photomicrographs: A—Pancreatin-containing digestive enzymes from Scientific Protein Laboratories (LotS1), B—Micropellets prepared by Powder layering in Granurex GX-35 (LotS2), C—Enteric coated micropellets of digestive enzymes (LotS3), and D—Enteric coated micropellets of digestive enzymes (LotS4).

FIG. 3: photomicrographs: A—Pancreatin-containing digestive enzymes from Nordmark and B—Micronized Pancreatin-containing digestive enzymes.

FIGS. 4A and B: respectively, photomicrographs of pancreatin particles (LotN1) before and after thermal treatment in cyclohexane (LotN2).

FIGS. 5A and B: photomicrographs of pancreatin microgranules (LotN3, 5% coacervated polymer).

FIGS. 6A and B: photomicrographs of pancreatin microgranules (LotN4, 20% coacervated polymer).

FIGS. 7A and B: photomicrographs of pancreatin microgranules (LotN5, 20% coacervated polymer).

FIGS. 8A and B: photomicrographs of selected pancreatin LotN1 (>250 μm).

FIGS. 9A and B: photomicrographs of pancreatin microgranules (LotN6, 5% coacervated polymer).

FIGS. 10A and B: photomicrographs of pancreatin microgranules (LotN7, 5% coacervated polymer).

FIGS. 11A and B: photomicrographs of selected pancreatin LotN1 (<250 μm).

FIGS. 12A and B: photomicrographs of pancreatin microgranules (LotN8, 20% coacervated polymer).

FIGS. 13A and B: photomicrographs of pancreatin microgranules (LotN9, 20% coacervated polymer).

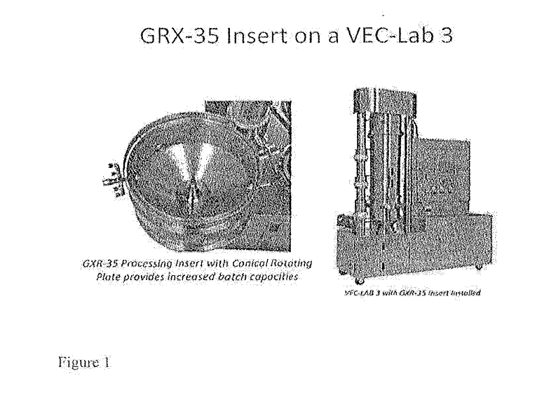

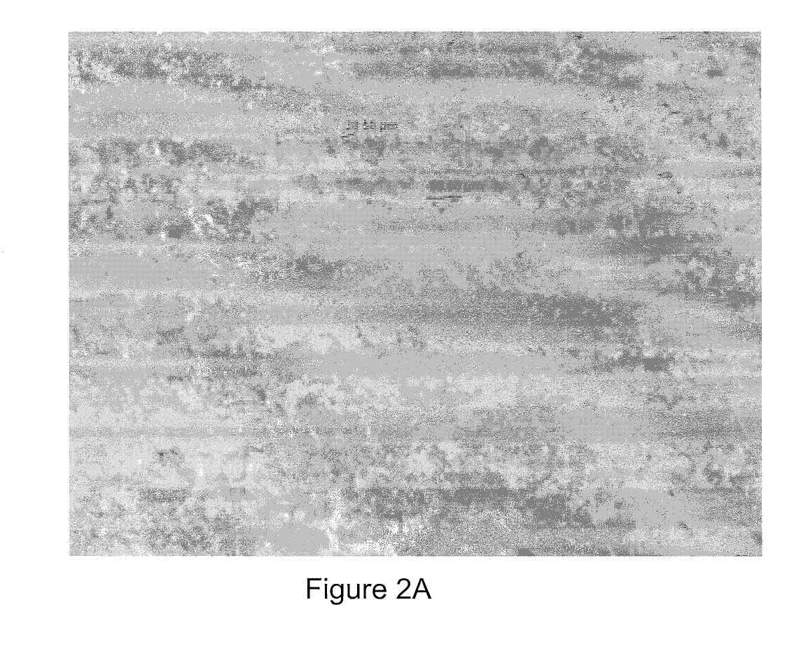

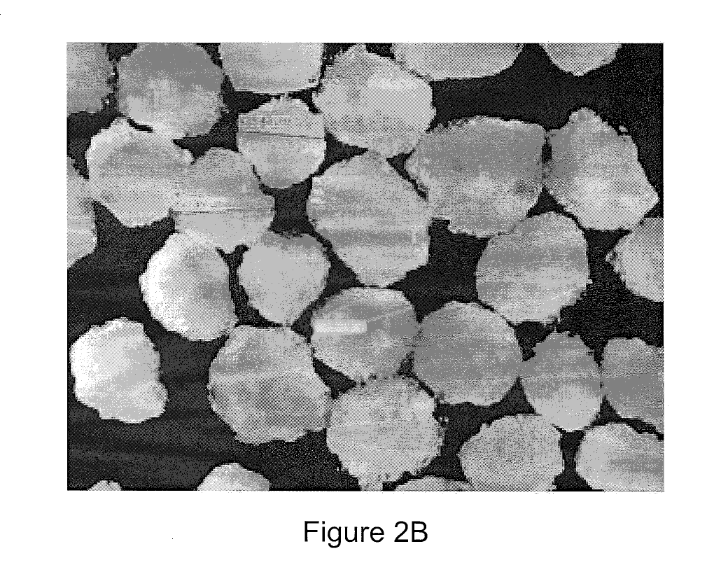

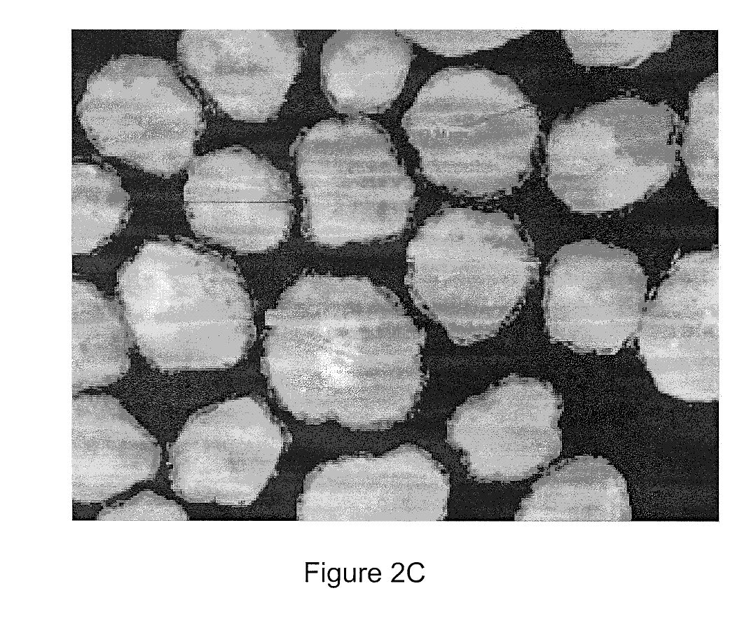

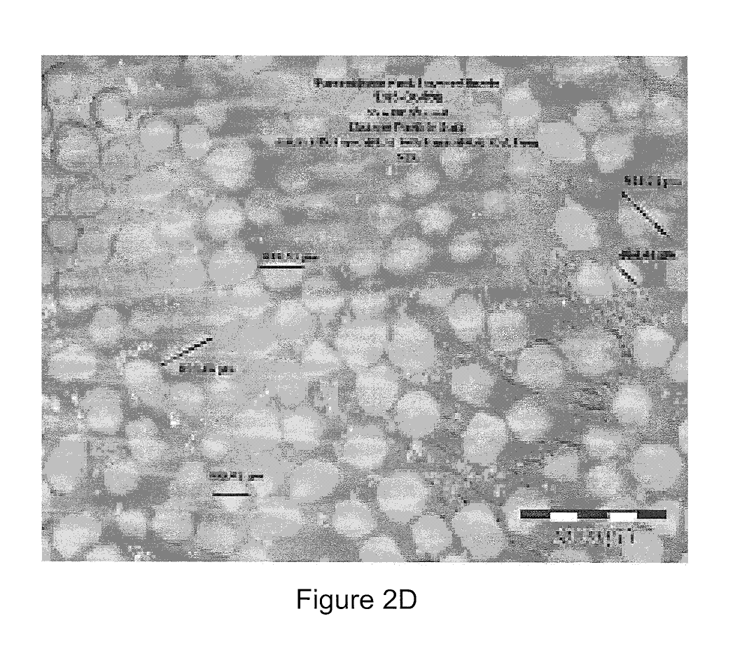

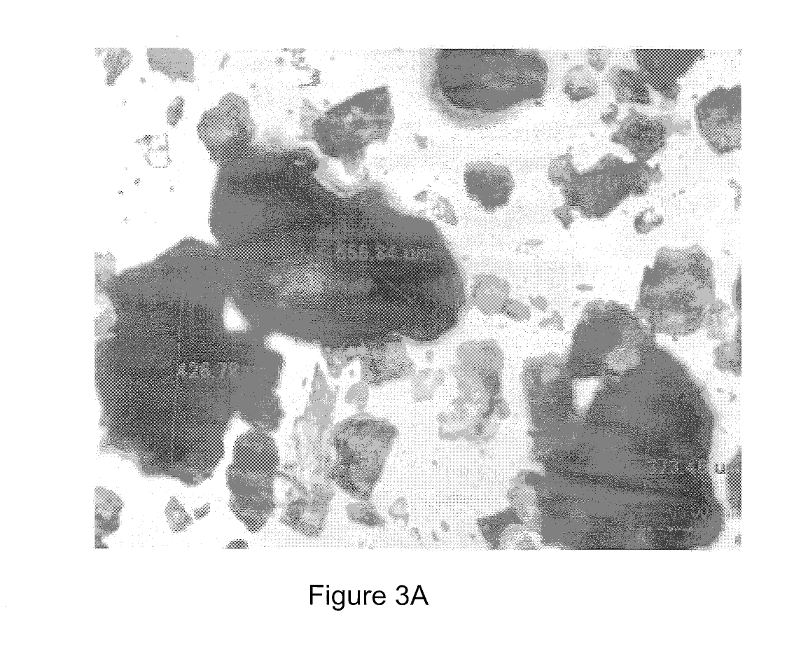

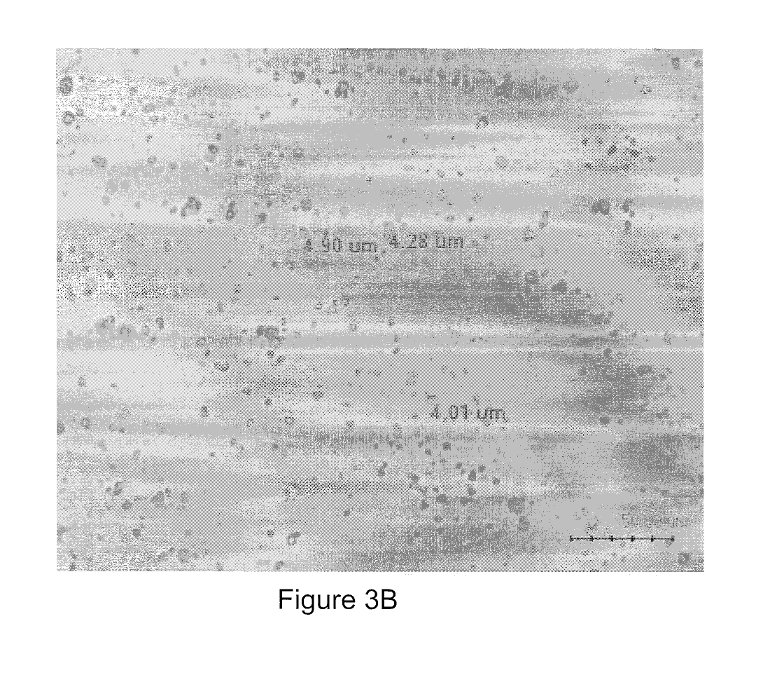

### DETAILED DESCRIPTION OF THE INVENTION

In various embodiments, the present invention relates to a small particle size composition comprising pancreatin containing digestive enzymes for use in patients in need thereof, including pediatric, geriatric, and adult patients, particularly those patients with dysphagia or wherein enteral administration using such composition would be suitable. In additional embodiments, the invention is directed to the composition in the form of particles, such as micropellets or microgranules having a high potency, high useable yield and at least 10%-90% of 400-800 μm particles. Furthermore, the composition optionally has an improved enteric coating and concomitant improved stability and enzyme activity compared to conventional prepared enterically coated pancreatic enzyme particles.

The present invention is directed to a pharmaceutical composition comprising, consisting essentially of, or consisting of a plurality of particles comprising at least one digestive enzyme, wherein said particles have low particle size dimensions. In particular, said particles have a volume diameter d(v,0.1) of not less than 400 μm and a volume diameter d(v,0.9) of no more than 800 μm as measured by laser diffraction or said particles have particle size of less than about 800 μm as measured by sieving. In certain embodiments they have a particle size ranging from about 200 μm to about 700 μm, from about 200 μm to about 600 μm, from about 400 μm to about 600 μm, or from about 250 μm to about 500 μm and all ranges and subranges therebetween as measured by sieving.

The particles comprise, consist essentially of, or consist of: 40-98 wt. % of digestive enzyme mixture; 2-20 wt. % of a polymer; optionally 0-30 wt. % of inert core; and optionally one or more pharmaceutically acceptable excipients. They may comprise at least 60% by weight of digestive enzyme mixture. They may have one or more coating layers.

The particles may be micropellets and they comprise, consist essentially of, or consist of 40-98 wt. % of digestive enzyme mixture; 2-10 wt. % of a water soluble polymer; optionally 0-30 wt. % of inert core; and optionally one or more pharmaceutically acceptable excipients. The micropellets may have one or more coatings layers. The polymer which is a component of the micropellets is a pharmaceutically acceptable water soluble polymer, which may be selected from the group consisting of hydroxypropyl-cellulose, povidone, methylcellulose, hydroxypropyl methylcellulose, carboxyalkylcellulose, polyethylene oxide, vinylpyrrolidone-vinyl acetate copolymer, polysaccharide, and mixtures thereof. Said micropellets may optionally comprise a hydrophobic water-insoluble polymer which may be selected from the group consisting of low viscosity ethylcellulose, cellulose acetate, cellulose acetate butyrate, polyvinyl acetate, neutral methacrylic ester copolymers, ammonio-methacrylate copolymers, and mixtures thereof.

The particles may be also microgranules and they comprise, consist essentially of, or consist of: 80-95 wt. % of digestive enzyme mixture; 5-20 wt. % of a water insoluble polymer; and optionally one or more pharmaceutically acceptable excipients. The water insoluble polymer of the microgranules is a coacervated polymer deposited on them. In certain embodiments of the invention, the water insoluble polymer is selected from ethylcellulose, cellulose acetate, cellulose acetate butyrate, polyvinyl acetate, neutral methacrylic ester copolymers, ammonio-methacrylate copolymers, and mixtures thereof.

The term “digestive enzyme” denotes an enzyme in the alimentary tract which breaks down the components of the food so that they can be absorbed by the organism. The term “stabilized digestive enzyme” means a digestive enzyme which maintains substantial enzyme activity after long term storage. The terms “pancrelipase” or “pancreatin” are also used herein; both terms denote a mixture of several types of enzymes, including amylase, lipase, and protease enzymes.

Non-limiting classes of digestive enzymes suitable for use in the present invention include lipases, amylases and proteases. Non-limiting examples of digestive enzymes include pancrelipase, lipase, trypsin, chymotrypsin, chymotrypsin B, pancreatopeptidase, carboxypeptidase A, carboxypeptidase B, glycerol ester hydrolase, phospholipase, phospholipase A2, sterol ester hydrolase, ribonuclease, deoxyribonuclease, alfa-amylase, papain, chymopapain, bromelain, ficin, beta-amylase, cellulase, beta-galactosidase, and mixtures thereof. They can be obtained through extraction from pancreas, produced artificially or obtained from sources other than pancreas such as form microbes, plants or other animal tissues.

Lipases are enzymes that catalyze the hydrolysis of lipids to glycerol and simple fatty acids. Examples of lipases suitable for the present invention include, but are not limited to animal lipase (e.g., porcine lipase), bacterial lipase (e.g., Pseudomonas lipase and/or Burkholderia lipase), fungal lipase, plant lipase, recombinant lipase (e.g., produced via recombinant DNA technology by a suitable host cell, selected from any one of bacteria, yeast, fungi, plant, insect or mammalian host cells in culture, or recombinant lipases which include an amino acid sequence that is homologous or substantially identical to a naturally occurring sequence, lipases encoded by a nucleic acid that is homologous or substantially identical to a naturally occurring lipase-encoding nucleic acid, etc.), synthetic lipase, chemically-modified lipase, and mixtures thereof.

Amylases are glycoside hydrolase enzymes that break down starch, for examples alfa-amylases, beta-amylases, acid alfa-glucosidases, salivary amylases such as ptyalin, etc. Amylases suitable for use in the present invention include, but are not limited to animal amylases, bacterial amylases, fungal amylases (e.g., Aspergillus amylase, for example, Aspergillus oryzae amylase), plant amylases, recombinant amylases (e.g., produced via recombinant DNA technology by a suitable host cell, selected from any one of bacteria, yeast, fungi, plant, insect or mammalian host cells in culture, or recombinant amylases which include an amino acid sequence that is homologous or substantially identical to a naturally occurring sequence, amylases encoded by a nucleic acid that is homologous or substantially identical to a naturally occurring amylase-encoding nucleic acid, etc.), chemically modified amylases, and mixtures thereof.

Proteases are generally enzymes (proteinases, peptidases, proteolytic enzymes) that break peptide bonds between aminoacids of proteins. Non-limiting examples of proteases suitable for use in the present invention include serine proteases, threonine proteases, cysteine proteases, aspartic acid proteases (e.g., plasmepsin) metalloproteases, and glutamic acid proteases. In addition, proteases suitable for use in the present invention include, but are not limited to animal proteases, bacterial proteases, fungal proteases (e.g., an Aspergillus melleus protease), plant proteases, recombinant proteases (e.g., produced via recombinant DNA technology by a suitable host cell, selected from any one of bacteria, yeast, fungi, plant, insect or mammalian host cells in culture, or recombinant proteases which include an amino acid sequence that is homologous or substantially identical to a naturally occurring sequence, proteases encoded by a nucleic acid that is homologous or substantially identical to a naturally occurring protease-encoding nucleic acid, etc.), chemically modified proteases, and mixtures thereof.

The compositions of the present invention can include one or more lipases (i.e., one lipase, or two or more lipases), one or more amylases (i.e., one amylase, or two or more amylases), one or more proteases (i.e., one protease, or two or more proteases), mixtures of one or more lipases with one or more amylases, mixtures of one or more lipases with one or more proteases, mixtures of one or more amylases with one or more proteases, or mixtures of one or more lipases with one or more amylases and one or more proteases.

In one embodiment, the digestive enzyme is a porcine pancreatic extract comprising various lipases (e.g., lipase, colipase, phospholipase A2, cholesterol esterase), proteases (e.g., trypsin, chymotrypsin, carboxypeptidase A and B, elastase, kininogenase, trypsin inhibitor), amylases, and optionally nucleases (ribonuclease, deoxyribonuclease). In another embodiment, the digestive enzyme is substantially similar to human pancreatic fluid. In yet another embodiment, the digestive enzyme is pancrelipase USP.

While porcine pancreatic enzyme preparations are widely used, the bovine enzyme preparations containing about 75% less lipase activity are an alternative for patients who do not consume porcine products. Microbial preparations of pancreatic enzymes (lipase, amylase, and protease) also exist. Certain bacteria (e.g., Burkholderia plantarii) and fungi (e.g., Aspergillus niger, Rhizopus arrhizus) have been shown to produce pancreatin enzymes with substantial lipolytic activity and greater resistance to gastric acid degradation.

Lipase activities in the compositions can be from about 2,500 to about 28,000 IU (USP method), from about 2,700 to about 3,300 IU, from about 4,500 to about 5,500 IU, from about 9,000 to about 11,000 IU, from about 13,500 to about 16,500 IU, and from about 18,000 to about 22,000 IU, from about 25,000 to about 27,500 IU and all ranges and subranges therebetween. Amylase activities in the compositions can be from about 6,000 to about 22,500 IU (USP), for example from about 6,400 to about 26,300 IU, from about 10,700 to about 43,800 IU, from about 21,500 to about 87,500 IU, from about 32,100 to about 131,300 IU, from about 42,900 to about 175,000 IU, from about 53,600 to about 218,700 IU and all ranges and subranges therebetween. Protease activities in the compositions can be from about 5,000 to about 130,000 IU (USP), for example from about 5,000 to about 15,400 IU, from about 8,400 to about 25,700 IU, from about 16,800 to about 51,300 IU, from about 25,000 to about 77,000 IU, from about 33,500 to about 102,800 IU, from about 41,800 IU to about 128,300 IU and all ranges and subranges therebetween.

In one embodiment, the lipase activity ranges from about 2,700 to about 3,300 IU, the amylase activity ranges from about 6,400 to about 26,300 IU, and the protease activity ranges from about 5,000 to about 15,400 IU (USP). In another embodiment, the lipase activity ranges from about 4,500 to about 5,500 IU, the amylase activity ranges from about 10,700 to about 43,800 IU, and the protease activity ranges from about 8,400 to about 25,700 IU (USP). In another embodiment, the lipase activity ranges from about 9,000 to about 11,000 IU, the amylase activity ranges from about 21,500 to about 87,500 IU, and the protease activity ranges from about 16,800 to about 51,300 IU (USP). In another embodiment, the lipase activity ranges from about 13,500 to about 16,500 IU, the amylase activity ranges from about 32,100 to about 131,300 IU, and the protease activity ranges from about 25,000 to about 77,000 IU (USP). In yet another embodiment, the lipase activity ranges from about 18,000 to about 22,000 IU, the amylase activity ranges from about 42,900 to about 175,000 IU, and the protease activity ranges from about 33,500 to about 102,800 IU (USP). In still another embodiment, the lipase activity ranges from about 25,000 to about 27,500 IU, the amylase activity ranges from about 53,600 to about 218,700 IU, and the protease activity ranges from about 41,800 IU to about 128,300 IU (USP).

In still another embodiment, the lipase activity ranges from about 5,000 PhEur lipase units to about 30,000 PhEur lipase units, it may be about 5,000, or about 10,000, or about 15,000 or about 20,000 or about 30,000 PhEur lipase units.

The ratio of amylase/lipase in the compositions can range from about 1 to about 10, in certain embodiments the ratio of amylase/lipase may be from about 2.38 to about 8.75 (enzymatic assay is performed according to USP). The ratio of protease/lipase in the compositions or oral dosage forms of the present invention can range from about 1 to about 8, in particular embodiments from about 1.86 to about 5.13 (enzymatic assay is performed according to USP).

The total amount of digestive enzymes (by weight) in the compositions or dosage forms of the present invention can be about 20-100%, about 40-100%, about 40-90%, about 40-80%, about 40-70%, about 40-60%, about 40-50%, and all ranges or subranges therebetween, or about 20%, about 30%, about 40%, about 50%, about 60%, about 70%, about 80%, about 90%, or about 100%. In one embodiment, the total amount of digestive enzymes is 40-80%. In another embodiment, the total amount of digestive enzymes (e.g., pancrelipase) is about 55-65%.

The term “particle includes but is not limited to a microparticle, microgranule, bead pellet, granulate, microspheres, powder etc., having a size typically in the range of from about 100 μm to about 800 μm including from about 200 μm to about 800 μm, about 200 μm to about 700 μm, about 200 μm to about 600 μm, about 200 μm to about 500 μm, about 200 μm to about 400 μm, about 250 μm to about 800 μm, about 250 μm to about 700 μm, about 250 μm to about 600 μm, about 250 μm to about 500 μm, about 300 μm to about 800 μm, about 300 μm to about 700 μm, about 300 μm to about 600 μm, about 300 μm to about 500 μm, about 400 μm to about 800 μm, about 400 μm to about 700 μm, about 400 μm to about 600 μm, about 400 μm to about 500 μm, about 700 μm to about 800 μm and all subranges therebetween as measured by sieving.

The particles (such as the micropellets and the microgranules) may have one or more coatings which may protect the enzymes from external environment (such as from moisture, protecting coating) or may act as separating film (sealing coating) or may modulate the drug release (functional coating). The coating composition comprises: one or more hydrophilic polymers, and one or more plasticizers, and optional pharmaceutically acceptable hydrophobic material, and mixtures thereof. Said hydrophobic material may have anti-tacking properties (e.g., talc, magnesium stearate, colloidal silicon dioxide, such as stearic acid, palmitic acid, or a fatty acid ester such as glyceryl monostearate, glyceryl palmitostearate, and mixtures thereof). Depending on the source of the digestive enzyme mixtures and intended use of the composition, the ratio of the hydrophilic polymer to the hydrophobic agent may be in a range of from about 10:1 to about 1:10 by weight. In another embodiment, the ratio of the hydrophilic polymer to the hydrophobic agent may range from about 5:1 to about 1:5 by weight. Non-limiting examples of the hydrophilic polymer include such as hydroxypropylcellulose, hydroxypropyl-methylcellulose, polyvinyl alcohol, vinyl pyrrolidone-vinyl acetate copolymer (e.g., Kollidon® VA 64 from BASF), some low viscosity ethylcellulose (e.g., viscosity of 10 cps or less a 5% solution in 80/20 toluene/alcohol at 25° C. as measured using an Ubbelohde viscometer), and mixtures thereof.

Non-limiting examples of suitable plasticizers include triacetin, tributyl citrate, tri-ethyl citrate, acetyl tri-n-butyl citrate, diethyl phthalate, dibutyl sebacate, polyethylene glycol, polypropylene glycol, castor oil, acetylated mono- and di-glycerides, cetyl alcohols, and mixtures thereof. In one embodiment the plasticizer is a non-phthalate plasticizer.

In one embodiment, the hydrophilic polymer comprising the sealing coating is hydroxypropylcellulose (e.g., Klucel LF) in combination with low viscosity ethylcellulose (e.g., Ethylcellulose Standard Premium 7 or 10) and the plasticizer is triethylcitrate. In another embodiment, the hydrophilic polymer comprising the coating is hydroxypropylcellulose, the plasticizer is triethylcitrate, and the anti-tacking agent is talc. In certain other embodiments, the hydrophilic polymer is a mixture of hydroxypropyl cellulose with low viscosity ethylcellulose, the plasticizer is tributyl sebacate, and the hydrophobic agent is glyceryl palmitate-stearate. The sealant coating can constitute from about 1% to about 20% of the weight of the drug-containing sealant-coated core, for example about 1%, about 2%, about 3%, about 4%, about 5%, about 7%, about 10%, about 12%, about 15%, about 17%, or about 20%, inclusive of all ranges and subranges there between.

The term “disposed over,” e.g. in reference to a coating over a substrate, refers to the relative location of e.g. the coating in reference to the substrate, but does not require that the coating be in direct contact with the substrate. For example, a first coating “disposed over” a substrate can be in direct contact with the substrate, or one or more intervening materials or coatings can be interposed between the first coating and the substrate. In other words, for example, a delayed release (DR) or enteric polymer coating disposed over the micropellets or the microgranules can refer to an enteric coating deposited directly over the particles, or can refer to a DR coating deposited onto a protective coating deposited on the particles. When the coating protects the drug from the gastroenteric environment than the coating composition comprises: one or more enteric polymers, and one or more plasticizers. This type of coating may be disposed over the uncoated micropellets or microgranules or over the coated (such as protective or sealing coated) particles.

The term “enteric polymer” means a polymer that protects the digestive enzymes from gastric contents, for example a polymer that is insoluble at acidic pH levels (e.g., of the stomach), but breaks down or dissolves at higher pH levels (e.g., in the lower gastrointestinal tract), or has a rate of hydration or erosion which is slow enough to ensure that contact of gastric contents with the digestive enzymes is relatively minor in the stomach, as opposed to the remainder of the gastrointestinal tract. The term “pH sensitive” as used herein refers to polymers which exhibit pH dependent solubility. The enteric coating comprises at least one enteric polymer and at least one plasticizer (e.g., hypromellose phthalate, HP-55 and triethyl citrate). In one embodiment, the ratio of the enteric polymer to the plasticizer ranges from about 7:3 to about 9:1 by weight. In another embodiment, the ratio of the enteric polymer to the plasticizer 9:1 by weight, and the enteric coating may range from about 10 wt. % to about 50 wt. % based on the weight of the coated micropellets or microgranules). The enteric coating ranges from about 25% to about 40% by weight, coating level may be about 10%, about 25%, about 30%, about 33%, about 35%, about 40%, about 50% by weight.

Examples of enteric polymers suitable for the present invention include, but are not limited to cellulose acetate phthalate, hydroxypropyl methylcellulose phthalate, hydroxypropyl methylcellulose acetate succinate, polyvinyl acetate phthalate, cellulose acetate succinate, pH sensitive methacrylic acid-methylmethacrylate copolymers (e.g., Eudragit S 100, Eudragit L 100, Eudragit FS, etc.), fatty acids, waxes, shellac, and mixtures thereof. In particular embodiments the enteric polymers are soluble in organic solvents.

Non-limiting examples of plasticizers suitable for incorporating into the enteric coating layer include triacetin, tributyl citrate, tri-ethyl citrate, acetyl tri-n-butyl citrate, cetylalcohol, diethyl phthalate, dibutyl sebacate, polyethylene glycol, polypropylene glycol, castor oil, acetylated mono- and di-glycerides, and mixtures thereof. In one embodiment the plasticizer is a non-phthalate plasticizer.

The enteric polymer can further comprise at least one inorganic agent, such as talc which may be in a range of from about 10:1 to about 1:60 by weight. In another embodiment, the ratio of the enteric polymer and the at least one inorganic material ranges from about 8:1 to about 1:50 by weight. In another embodiment, the ratio of the enteric polymer and the at least one inorganic material ranges from about 6:1 to about 1:40 by weight. In another embodiment, the ratio of the enteric polymer and the at least one inorganic material ranges from about 5:1 to about 1:30 by weight. In another embodiment, the ratio of the enteric polymer and the at least one inorganic material ranges from about 4:1 to about 1:25 by weight. In another embodiment, the ratio of the enteric polymer and the at least one inorganic material ranges from about 4:1 to about 1:9 by weight. In another embodiment, the ratio of the enteric polymer and the at least one inorganic material ranges from about 10:4 to about 10:7 by weight.

In still other embodiments, the enteric coating can further comprise at least one anti-tacking agent. Non-limiting examples of suitable anti-tacking agents include colloidal silicon dioxide, magnesium stearate, talc, glyceryl monostearate, and mixtures thereof. In one embodiment, the anti-tacking agent is talc. Depending on the enteric polymer or intended use of the composition, the ratio of the enteric polymer to the anti-tacking agent may be in a range of from about 10:0 to about 6:4 by weight. In another embodiment, the ratio of the enteric polymer to the antitacking agent ranges from about 9:1 to about 7:3 by weight.

Unless stated otherwise, the amount of the various coatings or layers described herein (the “coating weight”) is expressed as the percentage weight gain of the particles (micropellets or microgranules) provided by the dried coating, relative to the initial weight of the particles or beads prior to coating. Thus, a 10% coating weight refers to a dried coating which increases the weight of a particle by 10%.

The composition of the invention includes forming micropellets comprising at least one digestive enzyme with a median particle size of about 30 μm or less by controlled spheronization or powder layering in a suitable device (such as Granurex GX-35 from Vector Corporation), powder layering in a pan coater in accordance with the disclosures of U.S. Pat. No. 6,569,462, or alike, optionally coating the micropellets with a hydrophilic polymer solution optionally containing a hydrophobic anti-tacking agent. Both coated and uncoated micropellets, especially the micropellets comprising porcine-derived digestive enzyme mixtures are optionally coated with a gastroresistant coating comprising an enteric polymer and optionally a plasticizer.

The manufacture of small spherical micropellets with a smooth surface texture, whether by controlled spheronization or powder layering, requires the use of digestive enzymes with a median diameter of less than 30 μm, preferably smaller than 20 μm to achieve micropellets with a smooth morphology for coating with functional polymers. A laser particle size analyzer such as Malvern Instruments' Mastersizer is typically used to measure the particle size distributions of fine powders and granular microparticles by “dry cell” or “wet cell” method. A suspension of powder is measured with a low angle laser beam and the particle size distribution is calculated as specified in the user's manual (The Malvern Mastersizer Basic user manual; QS Small Volume Sample Dispersion Unit user manual). The volume median diameter d(v,0.5) defined as the diameter where 50% of the distribution is above and 50% is below. The volume diameter d(v,0.9), defined as the diameter where 90% of the volume distribution is below this value and 10% is above. The volume diameter d(v,0.1), defined as the diameter where 10% of the volume distribution is below this value and 90% is above this value. In particular embodiments of the invention, the particle size range for the micropellet cores is from about 400 μm to about 600 μm. The particle size distributions of fine powders and microparticles are also measured by the sonic sifter method using a stack of sieves.

Both controlled spheronization and powder layering processes require micronized drug particles (e.g., most desirable: d(v,0.5)<20 μm and d(v,0.9)<50 μm). In order to keep such fine powders within the product bed, both air velocity and total airflow need to be optimized/balanced and controlled during processing. Significant time savings are accomplished by adding the active in powder form rather than by dissolving or suspending in a polymer binder solution and spraying. The process results in higher useable yields with narrow particle size distributions.

During powder layering the rotor insert of a reactor (such as Granurex GX-35) is charged with inert cores such as sugar spheres of desired particle size. A fine powder mixture comprising the digestive enzymes, a flow-aid such as Cab-O-Sil (colloidal silicon dioxide) and optionally a polymer binder is fed into the machine via a precision powder feeder and dispersed directly onto inert cores while simultaneously spraying a polymer binder solution to bind the digestive enzyme powder onto the outside of the core material. Thus, the micropellet begins to grow layer by layer. Balancing the powder feed rate and spray rate is the most critical parameter to be achieved during powder layering, to avoid either overwetting and agglomeration or inefficient sticking of the powder onto inert cores resulting in lower yields. After all the free powder is bound up as micropellets, the spray is stopped and the micropellets are dried. Drying can be performed for example by either using slit air only or by dropping an air device called a Mov-A-Blo.

During controlled spheronization the rotor insert of suitable equipment (such as Granurex GX-35) is charged with a fine powder mixture comprising the digestive enzymes, a flow-aid such as Cab-O-Sil (colloidal silicon dioxide) and optionally a polymer binder. A polymer binder solution is sprayed onto finer powder bed at a controlled rate while simultaneously the powder is added into the unit via a powder feeder (K-Tron) at a controlled rate. The fine mist of the binder solution helps bind the digestive enzyme powder mixture onto micropellets which grow layer by layer. Again the process parameters such as air velocity, air flow, product temperature, spray rate are optimized/controlled, thereby avoiding over wetting/agglomeration or insufficient sticking of successive powder layers onto micropellets resulting in lower yields.

To the uncoated or sealed coated digestive enzyme containing micropellets a further enteric coating solution may be applied via fluid bed coating or pan coating or via Granurex GX-35 or alike.

Besides the controlled spheronization and the powder layering method, the small particle size particles comprising the digestive enzymes may be prepared by the coacervation process starting from pancreatin material having particle size between about 250 μm and 500 μm (as measured by sieving method). In this process, the digestive enzyme particles are treated with a solution comprising the water insoluble polymer dissolved in a hydrophobic organic solvent, such as cyclohexane. The water insoluble polymer solution further includes a phase separating agent. The process includes a heating the solution of the insoluble polymer, hydrophobic organic solvent and phase separation agent, and suspended digestive enzyme particles up to 80° C. and then cooling this mixture down to about 25° C. in the coacervation reactor. The phase separating agent includes polyethylene butyl rubber, polybutadiene, polyisobutylene, organosilicon polymers, and paraffin. This process does not affect the enzymatic stability of the enzymes. The obtained digestive enzymes microgranules have a low moisture content (below 3%) and have particle size of less than about 800 microns, including from about 200 to about 700 microns, from about 200 to about 600 microns, from about 250 to about 500 microns and all ranges and subranges therebetween (as measured by sieving). Said microgranules having coacervated polymer may be further coated with an enteric coating solution comprising an enteric polymer dissolved in a pharmaceutically acceptable solvent. The enteric coating step comprises coating the microgranules with an enteric coating solution by means of fluid bed coating or by means of pan coating. The preferred enteric polymer is soluble in organic solvent and is hydroxypropyl methylcellulose phthalate, the preferred solvent is acetone. The obtained enteric coated microgranules have a moisture content less than about 3%, preferably less than about 2%, they may have a moisture content of about 1.2% (measured by Loss on Drying (LoD) test, USP method).

The compositions and oral dosage forms of the present invention comprising low particle size dimensions particles with at least one digestive enzyme have a moisture content of about 4.5% or less, about 3% or less, about 2.5% or less, about 2% or less, about 1.5% or less, inclusive of all ranges and subranges therebetween (such as between about 1.5% and about 4.5%, between about 1.5% and about 1.5%, between about 1.5% and 2.5%). Compositions or oral dosage forms of the present invention maintain a low moisture content upon storage and are substantially more stable compared to conventional compositions maintained at higher moisture contents, e.g. above about 4% or higher. Determination of moisture content of dosage forms and compositions of the present invention is carried out by Loss on drying measurement (LoD, USP method).

The compositions and dosage forms of the inventions exhibits a loss of digestive enzyme activity of no more than about 25%, no more than about 20%, no more than about 15%, no more than about 12%, no more than about 10%, no more than about 8%, or no more than about 5%, after six months of accelerated stability testing; stability testing comprises storing the compositions in a sealed Nialene bag at 40° C., 75% relative humidity.

The term “accelerated stability testing” or “accelerated storage testing” refers to test methods used to simulate the effects of relatively long-term storage conditions on enzyme activity, which can be carried out in a relatively short time. Accelerated stability testing methods are known in the art to be a reliable alternative to real-time stability testing, and can accurately predict the shelf life of biological products. Such “accelerated stability testing” conditions are known in the art and are in accordance with the International Conference for Harmonization of Technical Requirements for Registration of Pharmaceuticals for Human Use: Stability Testing of New Drug Substances and Products Q1A, herein incorporated by reference.

The digestive enzyme compositions having small pancreatin particles (such as micropellets or microgranules) of the present invention may be used to prepare pharmaceutical dosage forms (e.g. capsules, sachets, and rapidly dispersing tablets) suitable for pediatric, geriatric, and/or patients with dysphagia, who experience difficulty swallowing conventional tablets/capsules. The dosage forms are suitable for oral and for enteral administration in patients. Dosage form such as the capsules may be swallowed or dispersed with food.

The capsules to be filled in with the small micropellets or microgranules may be hydropropyl-methylcellulose capsules, they have a moisture content of about 2% by weight or less, they may be dried prior to filling with the plurality of coated micropellets or microgranules.

The compositions of the present invention may also be formulated into sachets, for example mono-dose sachets, wherein the composition of the present invention is divided into single doses which are packaged separately, for example by introducing each dose into a sachet. In use, these sachets are dispensed into an aqueous medium such as water, thereby forming a suspension which is administered to the patient. Alternatively, the composition of the present invention can be dispensed from the sachet directly into the mouth, or can be dispensed directly onto food. When consumed directly or dispensed directly onto food, such mono-dose sachets are termed “dry sachets”.

Thus the compositions of the present invention in the different dosage forms can further comprise one or more pharmaceutically acceptable excipients. The term “excipients” includes other pharmaceutically acceptable ingredients blended with the particles comprising active component(s) of a composition (e.g., the digestive enzymes) in order to improve processing, stability, palatability, etc. Non-limiting examples of suitable excipients include pharmaceutically acceptable binders, sweeteners, flavors, stabilizers, disintegrants, lubricants, glidants, diluents, and mixtures thereof. It will be appreciated by those skilled in the art of pharmaceutical formulations that a particular excipient may carry out multiple functions in the composition. So, for example, a binder may also function as a filler.

Non-limiting examples of suitable lubricants include calcium stearate, magnesium stearate, sodium stearyl fumarate, stearic acid, zinc stearate, talc, waxes, glyceryl behenate, Sterotex®, Stearowet®, and mixtures thereof. Non-limiting examples of suitable glidants include colloidal silicon dioxide, talc, and mixtures thereof. Non-limiting examples of suitable stabilizers include trehalose, proline, dextran, maltose, sucrose, mannitol, polyols, silica gel, aminoguanidine, pyridoxamine, and mixtures thereof.

Non limiting examples of disintegrants include crospovidone (e.g., Polyplasdone XL, Polyplasdone XL-10), croscarmellose sodium (e.g., Ac-Di-Sol), sodium starch glycolate (e.g., Explotab, Explotab CV), and mixture of thereof such as microcrystalline cellulose and sodium starch glycolate or croscarmellose sodium and crospovidone.

The amount of disintegrant can be in the range of about any of about 0.1-30%, about 1%-30%, about 1%-25%, about 1%-20%, about 1%-15%, about 1%-10%, about 1%-5%, about 5%-10%, about 5%-15%, about 5%-20%, about 5%-25%, or about 5%-30% and all ranges and subranges therebetween. In one embodiment, the amount of disintegrant is about 2%-4%, or about 2%-3%, or about 2.5%.

Non-limiting examples of suitable diluents and fillers include anhydrous dibasic calcium phosphate, anhydrous dibasic calcium phosphate dihydrate, tribasic calcium phosphate, cellulose, lactose, magnesium carbonate, microcrystalline cellulose, microcrystalline cellulose, starch, calcium phosphate, lactose, sucrose, mannitol, sorbitol, and mixtures thereof. In one embodiment, the diluent is microcrystalline cellulose (e.g. Avicel). In another embodiment, the diluent is starch. In another embodiment, the diluent is lactose (e.g., Pharmatol). In another embodiment, the compositions of the present invention can comprise a combination of diluents such as microcrystalline cellulose, starch and lactose.

The amount of diluent can be in the range of about any of about 0.1-99%, about 1%-30%, about 1%-25%, about 1%-20%, about 1%-15%, about 1%-10%, about 1%-5%, about 5%-10%, about 5%-15%, about 5%-20%, about 5%-25%, or about 5%-30% and all ranges and subranges therebetween. In one embodiment, the amount of diluent is about 2%-5%, about 3%-5%, or about 4%.

The compositions of the present invention may be formulated as capsules by filling required amounts of uncoated micropellets or microgranules and/or gastroresistant polymer coated particles using methods known in the art. Alternatively, the digestive enzyme composition can be formulated as rapidly dispersing tablets. The step of forming the oral dosage form as a rapidly dispersing tablet (RDT) can comprise, for example, compressing a blend comprising said functional polymer coated micropellets or microgranules and at least one pharmaceutically acceptable excipient into the rapidly dispersing tablet form using a rotary tablet press and appropriate tooling with a logo and/or score if desirable. The rapidly dispersing tablets are suitable for oral administration in subjects or patients in need of medication for the treatment of a disease state by one of the modes of administration—(1) swallowing the whole tablet, (2) break the tablet into two halves for swallowing individually, and (3) disperse the tablet in about 150 mL water, swirl, and drink. The rapidly dispersing tablets formed thereby would provide rapid dispersion on contact with water or body fluids, gastroresistance until exiting from the stomach, and rapid, substantially-complete release of the digestive enzymes in the small intestinal tract. Either the compression mix is internally lubricated with a lubricant such as magnesium stearate or an external lubrication system (such as Matsui ExLube) may be used to lubricate the punches and dies prior to compression.

The compositions or dosage forms (e.g., tablets or capsules or sachet) of the present invention can be stored in a suitable package. The package should minimizes the ingress of moisture during transportation and/or storage, it is moisture proof and further contains desiccants; the package comprises a sealed container comprising a moisture resistant material; the desiccant (i.e., a substance which absorbs, reacts with, or adsorbs water, thereby being capable of reducing the humidity inside the package) and at least one dosage form are inside the sealed container. The moisture resistant material is selected from the group consisting of metal, glass, plastic, and metal coated plastic and the desiccant is selected from the group consisting of molecular sieve, clay, silica gel, activated carbon, and mixtures thereof. The term “moisture proof” refers to a package which has a permeability to water of less than about 0.5 mg water per cm³ of container per year.

The compositions of the present invention provide improved absorption of fats, proteins, and carbohydrates in patients suffering from conditions or disorders associated with a digestive enzyme deficiency. In one embodiment, compositions of the invention, in particular pancrelipase or pancreatin compositions, may be used to treat exocrine pancreatic insufficiency (EPI) associated with various diseases. Such diseases include, but are not limited to cystic fibrosis (CF). In some embodiments, such compositions may substantially alleviate malabsorption (e.g. of fats) associated with EPI in cystic fibrosis patients and other patients, including pediatric patients. In some embodiments, such compositions may increase the coefficient of fat absorption (CF-A) to at least about 85% or more in cystic fibrosis patients. Such results may be achieved with or without co-administration with other agents or compositions. In one embodiment, such CF-A results are achieved without co-administration of proton pump inhibitors.

For patients identified as having low GI pH levels (e.g., GI pH levels<about 4), improved results may be obtained by administering the compositions or dosage forms of the present invention together with proton pump inhibitors, antacids, and other drugs which increase the pH of the GI tract. For example, the compositions or dosage forms of the present invention can be administered separately from the proton pump inhibitors, antacid, or other drugs (either prior to, concurrently with, or after administration of the proton pump inhibitor, antacid, etc.). Alternatively, the proton pump inhibitor, antacid, or other drug can be combined with the pancreatin composition of the present invention as a single dosage form.

In yet another embodiment, the present invention provides a method of treating or preventing a patient with a disorder associated with a digestive enzyme deficiency comprising administering a composition of the present invention comprising uncoated particles comprising digestive enzyme mixtures of animal, microbial, fungal, bacterial origin, particles comprising porcine-derived digestive enzyme mixtures coated with a gastroresistant polymer, or a mixture of both uncoated and enteric coated particles, to a mammal in need thereof. In one embodiment, the mammal is a human.

In yet another embodiment, the present invention provides a method of treating or preventing a patient with a disorder associated with a digestive enzyme deficiency comprising administering a composition or dosage form of the present invention to a patient in need thereof; wherein the composition or dosage form of the present invention comprises, in addition to at least one digestive enzyme, a proton pump inhibitor, antacid, or other medicament which increases GI pH. In still another embodiment, the present invention provides a method of treating a patient or preventing a disorder associated with a digestive enzyme deficiency, comprising administering a composition or dosage form of the present invention, in combination with a dosage form comprising a proton pump inhibitor, antacid, or other medicament which increases GI pH.

Disorders which can be treated with the composition or dosage form of the present invention include conditions in which the patient has no or low levels of digestive enzymes or in which patients require digestive enzyme supplementation. For example, such conditions can include cystic fibrosis, chronic pancreatitis, other pancreatic diseases (e.g., hereditary, post-traumatic and allograft pancreatitis, hemoehromatosis, Shwachman syndrome, lipomatosis, or hyperparathyroidism), side-effects of cancer or cancer treatment, side-effects of surgery (e.g., gastrointestinal bypass surgery, Whipple procedure, total pancreatectomy, etc.) or other conditions in which pancreatic enzymes cannot reach the intestine, poor mixing (e.g., Billroth II gastrectomy, other types of gastric by-pass surgery, gastrinoma, etc.) side effects of drug treatments such as treatment with metformin or those drugs used to treat the symptoms of HIV and autoimmune diseases such as diabetes in which the pancreas may be compromised, obstruction (e.g., pancreatic and biliary duct lithiasis, pancreatic and duodenal neoplasms, ductal stenosis), malabsorption associated with celiac disease, food allergies and aging.

The amount of the composition or dosage form of the present invention administered daily to mammals (e.g., humans) depends upon the intended result. The skilled physician will be capable of prescribing the required dose based on his diagnosis of the condition to be treated.

For example, for the treatment of digestive enzyme insufficiency in humans (e.g., related to cystic fibrosis) the starting dose should be 500 to 1000 lipase units/kg/meal, with the total dose not exceeding 2500 lipase units/kg/meal or 4000 lipase units/g fat/meal in accordance with the recommendations of the US FDA. Typically, a patient should receive at least 4 dosage forms per day, preferably administered with food.

All references cited herein are incorporated by reference in their entirety for all purposes.

### EXAMPLES

### In Vitro Potency/Gastroresistance/Dissolution Testing of Micropellets of Examples 1-3

Lipase activity is determined by a validated titration method using a potentiometric titrator equipped with a microburette, calomel-glass electrode system, resistance thermometer. USP Pancreatin Lipase RS or Pancrelipase working standard. Amylase activity is also determined by a validated titration method while protease activity is determined by a validated UV detection test method. Identification is achieved through a comparison of the chromatographic patterns (e.g., peaks of test enzymes and reference/working standard at certain relative retention times characteristic of lipase, amylase, and protease) obtained with a qualified RP-HPLC method. The peak areas of characteristic peaks between the test and reference/working standard must satisfy present specifications. The percentage of digestive enzyme dissolved when tested for dissolution in accordance with the current USP General Chapter <711> using USP Apparatus 1 (basket at 100 rpm) in 800 mL of simulated gastric fluid TS, without enzyme at 37±0.5° C. for 60 min (gastric resistance) and thereafter in 800 mL of pH 6.0 phosphate buffer (obtained by dissolving 2 g of sodium chloride and 9.20 g of monobasic phosphate in 1000 ml of water, and adjust with 1N sodium hydroxide to a pH of 6.0±0.05) for 30 min (to meet dissolution specification of) and the samples pulled at 60 and 90 min are tested for lipase activity using a validated potentiometric titration method.

Loss on Drying test (LoD) is performed according to the current USP chapter <731>; it is performed on 2 g of ground sample; the loss on drying is determined after drying in an oven under vacuum at 60° C. for 4 hours.

Free Phthalic Acid Content: the free phthalic acid content is detected/quantified by a qualified HPLC method (UV detection) in comparison to reference phthalic acid.

### Example 1

### Example 1.A

### Pancrelipase Micropellets by Powder layering

Povidone (PVP K-30; 50 g) is slowly added to 50/50 isopropanol/purified water (500 g) while constantly stirring to prepare a polymer binder solution at 10% w/w solids. Pancrelipase (LotS1) from Scientific Protein Laboratories (2000 g) is blended with 10 g of colloidal silica (a flow aid, Syloid from WR Grace Co.) and povidone (50 g) in a V-blender. Sugar spheres (60-80 mesh or 170-250 μm in diameter) are charged into the product bowl of Granurex GX-35 from Vector Corporation (Iowa, USA). The 10% PVP binder solution is sprayed into the rotating material bed at a controlled rate while feeding the digestive enzyme powder mixture at a controlled rate via the powder feeder. Optimized process parameters during pellet formation—process air temperature: about 19-20° C.; product temperature: 16±2° C.; rotor speed: 425 RPM; external air supply: 150 L/min; spray rate: 15 RPM (about 8 mL/min); pressure drop across slit: 1.3-11 mm in water. Optimized process parameters during drying of pellets—process air volume: 30 CFM; process air temperature: about 60° C.; product temperature: 35° C. (to stop drying); rotor speed: 180 RPM; slit air volume: 10 CFM; processing time: 40 min. Micropellets thus produced (LotS2) had a moisture content (loss on drying) of 4.1% while typical LoDs of incoming raw material lots are 1.5-2.0%. Composition of the micropellets: pancreatin: 82.5%; sugar spheres: 14.0% and povidone: 3.5%. Particle size distribution—d(0.1): 370 μm; d(0.5): 520 μm; d(0.9): 733 μm as measured by a Malvern laser particle sizer. The particle size distribution data measured using a sonic sifter is presented in Table 1 below.

TABLE 1
Particle Size Distribution of Pancreatin Micropellets (LotS2)

| Mesh Size | Microns | % Retained |
|---|---|---|
| 14 | 1400 | 0.0 |
| 20 | 850 | 0.3 |
| 30 | 600 | 10.1 |
| 40 | 425 | 74.1 |
| 50 | 300 | 15.4 |
| 80 | 180 | 0.2 |
| Pan | | 0 |

### Example 1.B

### Gastroresistant Coating of Micropellets (LotS3; Weight Gain: Up to 45 Wt. %)

Hypromellose phthalate (HP-55, 785.16 g) is slowly added to a 90/10 mixture of acetone and water while stirring constantly until dissolved followed by the addition of diethyl phthalate (DEP; 196.15 g), until the plasticizer dissolved. A Glatt GPCG 3 equipped with a 7″ bottom spray Wurster 22 mm high column, partition column gap of 15 mm from the ‘D’ bottom air distribution plate covered with a 200 mesh product retention screen (1.0 mm port nozzle) is charged with the micropellets from Example 1.A above, and are coated with the hypromellose phthalate solution (15% solids) at a product temperature of 37±1° C., atomization air pressure of 1.5 bar, inlet air velocity of 50-60 m/s, and a spray flow rate of 15-40 mL/min for a DR coating level of 45% by weight. Samples are pulled at a coating level of 25%, 30%, 35%, and 40%. Micropellets at a coating level of 35 wt. % and 45 wt. % are tested for gastroresistance. Based on the data, a delayed release coating of 35% w/w is judged to be sufficient to impart gastroresistant properties on the micropellets.

### Example 1.C Gastroresistant Coating of Micropellets (Weight Gain: 35 Wt. %)

A batch of micropellets gastroresistant coated with HP-55/DEP at a coating level of 35 wt. % (LotS4) is prepared. Process conditions—product temperature: 37-42° C.; process air velocity: about 5-60 m/s. The coated batch had a LoD of 2.4% upon drying at 105° C. Delayed release pancreatin micropellets had a particle size distribution with d(0.1): 445 μm; d(0.5): 605 μm; d(0.9): 827 μm).

### Example 1.D

### Stability Testing of DR Coated Micropellets (LotS3)

The tests for identification, confirmation, potency, gastroresistance, and complete release at pH of 6.0 of lipase, amylase, and protease enzymes including reference standards, and gastroresistance coated micropellets of the digestive enzymes are performed as discussed earlier. The initial test results are given in Table 2 below. Since more than 95% is released (detected) in 30 min, the gastroresistance testing is not considered necessary. The hermetically sealed glass bottles containing delayed release micropellets are placed on stability at 25° C./60% RH and 40° C./75% RH.

TABLE 2
Gastroresistant Micropellets (LotS4)

| | Lipase | Protease | Amilase | Gastro- | Dissolution | Loss on | Phthalic |
|---|---|---|---|---|---|---|---|
| Batch | (USP U/mg) | (USP U/mg) | (USP U/mg) | resistance (%) | (%) | Drying | Acid |
| LotS4 | 65 | 217 | 135 | NT | 97% | 2.8% | 0.2% |

### Example 1.E

### Gastroresistant Coating of Micropellets (Weight Gain: 28 Wt. %)

A batch of micropellets gastroresistant coated with a solution of HP-55/DEP at 80/20 in 100% acetone containing 10 g talc for a coating level of 28 wt. % using micropellets (LotS2) is also prepared. Process conditions—product temperature: 31-33.52° C.; process air volume: 65-70 CFM; spray rate: 15-30 g/min.

### Example 2

### Example 2.A

### Pancrelipase Micropellets by Powder layering

Povidone (PVP K-30; 50 g) is slowly added to isopropanol (650 g) while constantly stirring to prepare a polymer binder solution at 7% w/w solids. Pancrelipase (LotS1) from Scientific Protein Laboratories (2000 g) is blended with 10 g of colloidal silica (a flow aid, Syloid from WR Grace Co.) and povidone (50 g) in a V-blender. Sugar spheres (60-80 mesh or 170-250 μm in diameter) are charged into the product bowl of Granurex GX-35 from Vector Corporation (Iowa, USA). The 7% PVP binder solution is sprayed into the rotating material bed at a controlled rate while feeding the digestive enzyme powder mixture at a controlled rate via the powder feeder. Optimized process parameters during pellet formation—process air temperature: about 19-20° C.; product temperature: 16+2° C.; rotor speed: 425 RPM; external air supply: 150 L/min; spray rate: 15 RPM (about 8 mL/min); pressure drop across slit: 1.3-11 mm in water. Optimized process parameters during drying of pellets—process air volume: 30 CFM; process air temperature: about 60° C.; product temperature: 35° C. (to stop drying); rotor speed: 180 RPM; slit air volume: 10 CFM; processing time: 40 min.

### Example 2.B

### Gastroresistant Coating of Micropellets (Weight Gain: 30 Wt. %)

The micropellets from Ex. 2.A above are coated with the gastroresistant polymer HP-55/TEC/talc (ratio: 10/1/5) at a coating level of 30 wt. % (LotS5) in the Granurex GX-35. Hypromellose phthalate (HP-55) is slowly added into acetone in a stainless steel tank while constantly stirring to dissolve. Triethyl citrate (TEC) is added to the solution to dissolve, and talc (an alkalinizing agent) is added to the coating solution to achieve a homogeneous dispersion. Process conditions—product temperature: 37-42° C.; process air velocity: about 5-60 m/s.

### Example 3

### Example 3.A

### Pancrelipase Micropellets by Controlled Spheronization

Povidone (PVP K-30; 50 g) is slowly added to 90/10 isopropanol/purified water while constantly stirring to prepare a polymer binder solution at 7% w/w solids. Pancrelipase powder from SPL (2000 g) is blended with 10 g of colloidal silica (a flow aid, Cab-O-Sil M-5P from Cabot Corporation) and povidone (90 g) in a V-blender and charged into the product bowl of Granurex GX-35 from Vector Corporation (Iowa, USA). The 7% PVP binder solution is sprayed into the rotating material bed at a controlled rate while simultaneously the powder is added into the unit with a powder feeder (K-Tron) at a controlled rate. Optimized process parameters during pellet formation—process air temperature: about 19-20° C.; product temperature: 16+2° C.; rotor speed: 180 RPM; slit air volume: 10 CFM; spray rate: 15 RPM (about 8 mL/min); pressure drop across slit: 1.3-11 mm in water; processing time: 40 min.

### Example 3.B

### Moisture Resistant Coating of Micropellets

Klucel LF is slowly added to dehydrated ethanol in a stainless steel tank while constantly stirring to dissolve. Ethylcellulose (EC-10) is slowly added to the Klucel solution while constantly stirring to dissolve. Magnesium stearate (Mgst) is added to the coating solution to achieve a homogeneous dispersion. A Glatt GPCG 3 equipped with a 6″ bottom spray Wurster 8″ high column, partition column gap of 15 mm from the ‘D’ bottom air distribution plate covered with a 200 mesh product retention screen (1.0 mm port nozzle) is charged with the micropellets (1200 g) from Example 3.A, above and coated with the protective moisture resistant coating solution (10 wt. % solids of Klucel/EC-10/Mgst at a ratio of 30/45/25) at 5 mL/min, ramping up to about 20 mL/min.

### Example 3.0

### Gastroresistant Coating of Micropellets

The micropellets from Ex. 3A, above are coated with a gastroresistant coating of HP-55/TEC (ratio: 90/10) at a coating level of 30 wt. % in a Glatt GPCG 3 as disclosed above.

### Example 4

### Example 4.A

### Moisture Resistant Coating of Micropellets

Klucel LF is slowly added to purified water while constantly stirring to dissolve. Ethylcellulose (EC-10) is slowly added to the Klucel solution while constantly stirring to dissolve. Micropellets (1200 g) from Example 3.A, above are coated with the protective moisture resistant coating solution (10 wt. % solids of Klucel/EC-10 at 40/60) for a 10% weight gain.

### Example 4.B

### Gastroresistant Coating of Micropellets

The micropellets from Ex. 3A, above are coated with a gastroresistant coating of HP-55/TEC (ratio: 90/10) at a coating level of 30 wt. % in a Glatt GPCG 3 as disclosed above.

### Example 4.C

### Modified Release Digestive Enzyme Capsules

Moisture-resistant coated micropellets from Ex. 3.B and gastroresistant coated micropellets from Ex. 4.B, above are filled at a digestive enzyme weight ratio of 1:1 into HPMC capsules to produce MR capsules comprising digestive enzymes. The HPMC capsules are packaged in hermetically sealed glass bottles optionally containing molecular sieve type desiccants.

### Example 4.D

### Rapidly Dispersing Tablets of Digestive Enzymes

75-85 parts of gastroresistant coated micropellets from Ex. 4.B, above are blended with 10-15 parts of spray dried mannitol, 10-15 parts of microcrystalline cellulose (Avicel PH102), 2-7 parts of low-substituted HPC (disintegrant), and 0.2-0.5 parts of FD&C Red are first blended in a V blender for 20 minutes, and then 0.5 part of sodium stearyl fumarate is added to the blender to homogeneously distribute the lubricant by blending for about 10 minutes. The compression blend thus produced is compressed into 500 or 1,000 mg RDTs (rapidly dispersing tablets) using a rotary tablet press.

### Example 5

### Example 5.A

### Micronization of Pancrelipase

The pancrelipase from Nordmark Arzneimittel GmbH with a d(0.95) of 115 μm is micronized under nitrogen purging at a medium energy level at JET PHARMA to have a d(0.95) of 22.8 μm.

### Example 5.B

### Powder Layering via Gravity Feeder

A ventilated coating drum (Pellegrini GS), equipped with powder feeder, aircap spraying system, and peristaltic pump, is set-up. The micronized pancreatin as disclosed in Ex. 5.A above is blended with lactose and colloidal silicon dioxide. The polymer hinder and Tween 80 are dissolved in a suitable solvent mixture in a stainless steel container while constantly stirring. Sugar spheres (60-80 mesh) preheated at 30° C. in the ventilated coating drum rotating at 15 RPM is layered with discontinuous cycles of layering of pancreatin powder mix via a gravity feeder, binder solution spraying, and drying at an inlet temperature setting of 40° C. and airflow setting of about 250-300 m³/hour. Once the drug loading is complete, the micropellets are dried to drive off residual solvents, cooled to room temperature, and sieved using appropriate sieves to discard oversized (i.e., >700 μm) and finer material (i.e., <300 μm).

### Example 5.0

### Powder Layering Via Electrostatic Feeder

A ventilated coating drum (Pellegrini GS), equipped with powder feeder connected with an electrostatic gun, aircap spraying system, and peristaltic pump, is set-up. The micronized pancreatin as disclosed in Ex. 5.A above is blended with lactose and colloidal silicon dioxide. The polymer binder and Tween 80 are dissolved in a suitable solvent mixture in a stainless steel container while constantly stirring. Sugar spheres (60-80 mesh) preheated at 30° C. in the ventilated coating drum rotating at 15 RPM is layered by simultaneously feeding the pancreatin powder mix via the electrostatic feeder, spraying the binder solution, and drying at an inlet temperature setting of 45° C. and airflow setting of about 250 m³/hour. Once the drug loading is complete, the micropellets are dried to drive off residual solvents, cooled to room temperature, and sieved using appropriate sieves to discard oversized (i.e. >700 μm) and finer material (i.e. <300 μm).

The micropellets from Ex. 5.B or Ex. 5.0 are further coated with a moisture-resistant and/or gastroresistant coating as disclosed previously to impart moisture resistant and/or gastroresistant properties. Required amounts of uncoated or optionally moisture-resistant coated micropellets and/or gastroresistant coated micropellets may be filled into HPMC or gelatin capsules or sachets or blended with pharmaceutically acceptable excipients and compressed into rapidly dispersing tablets to produce desired modified release digestive enzymes for oral administration in patients in need of medication.

### Example 6

### Characterization of Starting Pancreatin Material

The appearance, enzymatic activity, and protease dissolution rate of pancrelipase starting material—Pancreatin LotN1 are analysed. The dissolution rate test is carried out at pH 6.0 and protease is chosen as model enzyme for dissolution rate microgranules evaluation instead of lipase which degrades in the dissolution medium during the 30 minute-long test procedure (after about 5 minutes, the lipase levels dropped to only 80%, and decreased down to 73% after 30 minutes). The protease dissolution rate and enzymatic activity (measured with the method based on the United States Pharmacopoeia, Pancrelipase: Assay for Protease activity) are shown below in Table 3. The particle size distribution is shown in Table 4.

TABLE 3
Dissolution Rate and Enzymatic Activity of LotN1

| | Dissolution rate (%) minutes | | | Assay |
|---|---|---|---|---|
| Pancreatin | 5 min. | 15 min. | 30 min. | U USP/mg |
| Pancreatin-LotN1 | 104 | 103 | 101 | 345 |

TABLE 4
Particle Size Distribution of Pancreatin LotN1 (sieving method)

| | % of particles within indicated |
|---|---|
| PSD Sieves | size range |
| <600 μm | 100% |
| >425 μm | 1% |
| >150 μm | 70% |
| <150 μm | 29% |

### Example 7

### Effect of Thermal Cycles on Pancreatin in Cyclohexane

The stability of pancreatin LotN2 to the thermal and mechanical stresses produced during a coacervation process carried out in cyclohexane is tested: 135 g of pancreatin is placed in a reactor containing 1 kg of cyclohexane and, under continuous stirring (290 RPM), heated and cooled (Table 5).

TABLE 5
Thermal Cycle Coacervation

| Phase | Temperature range (° C.) | Time (minutes) |
|---|---|---|
| Heating | 25-80 | 25 |
| Cooling | 80-65 | 15 |
| | 65-50 | 30 |
| | 50-25 | 32 |

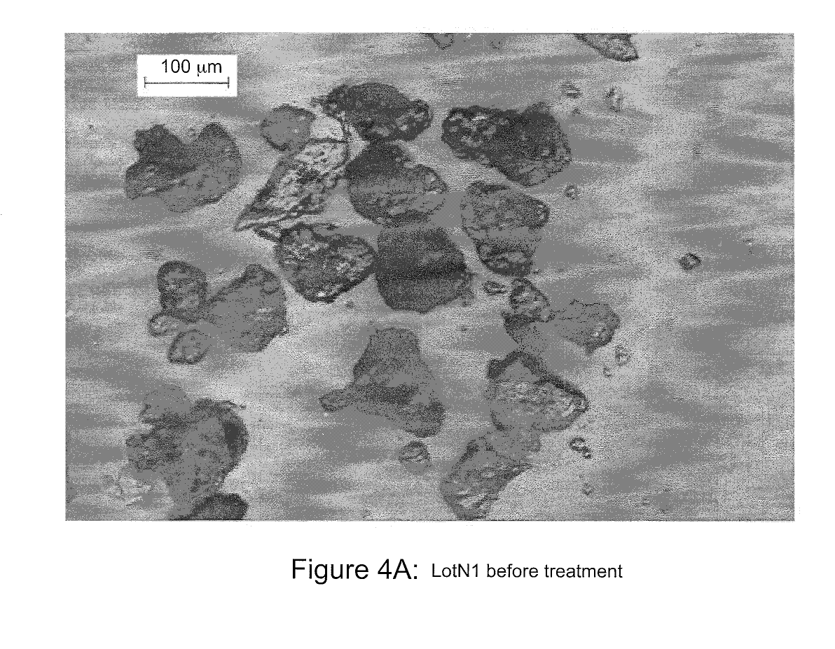

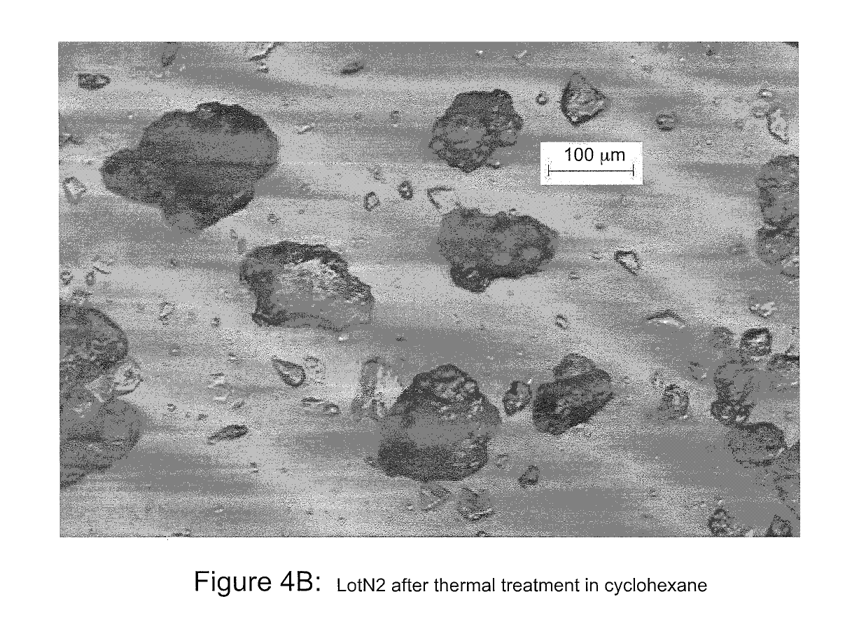

After being subjected to the thermal cycle, the pancreatin is filtered and dried overnight (approximately 16 h) in a fume hood. The resulting powder is sieved through a 500 μm sieve and stored in a high density polyethylene (HDPE) container. Photomicrographs of pancreatin LotN1 are taken with a Zeiss Axioscopic microscope before (FIG. 4A) and after thermal treatment in cyclohexane (FIG. 4B; LotN2), do not show evidence of any significant change in appearance, shape and dimension of the particles. The analytical results confirm the microscopic observations (Table 6).

TABLE 6
Enzymatic Activity Values and Protease Dissolution Rate -
Before and After Thermal Cycle in Cyclohexane

| | Pro- | Li- | Cyclo- | | DRT pH 6.0 Protease | |
|---|---|---|---|---|---|---|
| Pancreatin | tease | pase¹ | hexane² | LoD | 5 | 30 |
| LotN1 | (U USP/mg) | | (ppm) | (%) | min. | min. |
| Before | 345 | 85 | — | n.d. | n.d. | n.d. |
| Treatment | | | | | | |
| Before | 308 | 82 | — | n.d. | n.d. | n.d. |
| Treatment + | | | | | | |
| 16 h hood | | | | | | |
| After | 315 | 81 | 13 | 5.5% | 100% | 100% |
| Treatment + | | | | | | |
| 16 h hood | | | | | | |

¹Lipase activity: tested using conventional method based on the United States Pharmacopoeia, Pancrelipase: Assay for lipase activity.
²Residual cyclohexane: measured by gas chromatography (GC equipped with a headspace sampler device and a flame-ionization detector).

Protease and lipase activities of LotN2 do not show significant changes before and after being subjected to the thermal cycle in cyclohexane. The residual amount of cyclohexane, after 16 hours of drying in a fume hood at room temperature, is negligible, and it is confirmed that pancreatin tends to absorb water. Protease dissolution values reach 100% within 5 minutes and do not appear to decrease during the test.

### Example 8

### Preparation of Pancreatin Microgranules

1000 g of cyclohexane is poured into a coacervation reactor. Then, under continuous stirring (290 RPM), pancreatin, ethylcellulose (amounts ranging from 0.4% to 5%) and polyethylene (amounts ranging from 0% to 3%) are added. The resulting mixture is heated and cooled according to the thermal cycle of Table 5. The resulting pancreatin microgranules are washed (one or more times), filtered and dried overnight (about 16 h) in a fume hood, then sieved through a 500 μm opening sieve and stored in a HDPE container. The process flow sheet is provided below:

| COMPONENT | STEP | EQUIPMENT |
|---|---|---|
| Pancrelipase | Coacervation/Phase | Reactor |
| Ethylcellulose | Separation | Thermocryostate |
| Epolene¹ | | Stirrer |
| Cyclohexane² | | |
| | Washing | Filtering system |
| | Filtering | Filtering system |
| | Drying | Hood, Oven |
| | Sieving | Sieve |

¹Removed during washing step
²Removed during drying step

Three batches of microgranules are prepared using essentially this procedure. The number of washes, ethylcellulose and polyethylene concentrations, and the coating weight of the coacervated ethylcellulose (% w/w; weight gain of the microgranules relative to the weight of the starting pancreatin microgranules) in the finalproduct are summarized in Table 7.

TABLE 7
Coacervated Polymer Weight, Ethylcellulose Conc.,
Polyethylene Conc., and No. of Washes

| | Coacervated | | | |
|---|---|---|---|---|
| | Polymer | Ethylcellulose | Polyethylene | |
| Lot | Weight (%) | Concentration | Concentration | No. Washes |
| LotN3 | 5 | 0.9% | 0.5% | None |
| LotN4 | 20 | 2% | 0.5% | None |
| LotN5 | 20 | 2% | 2% | 3 |

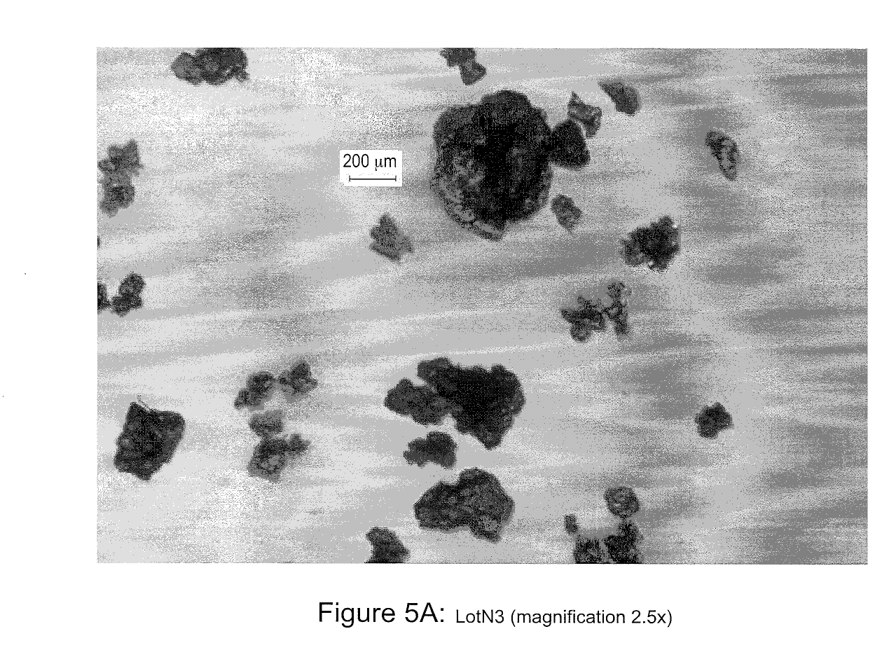

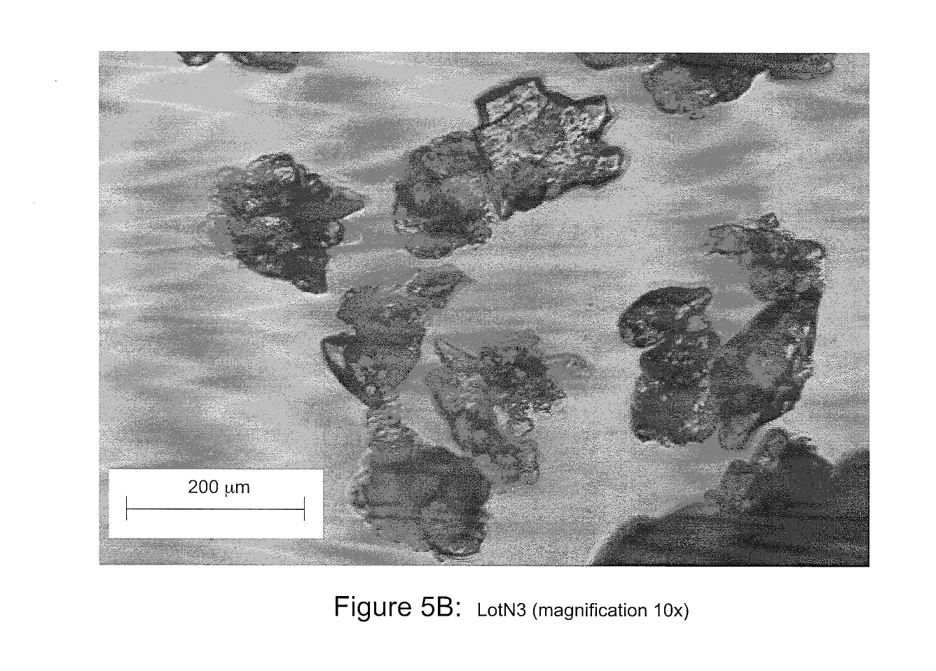

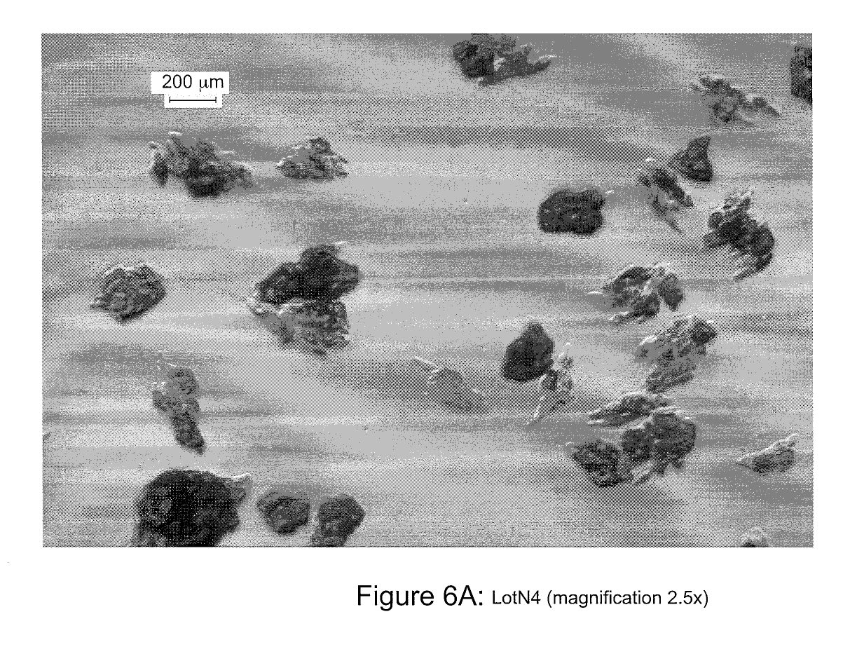

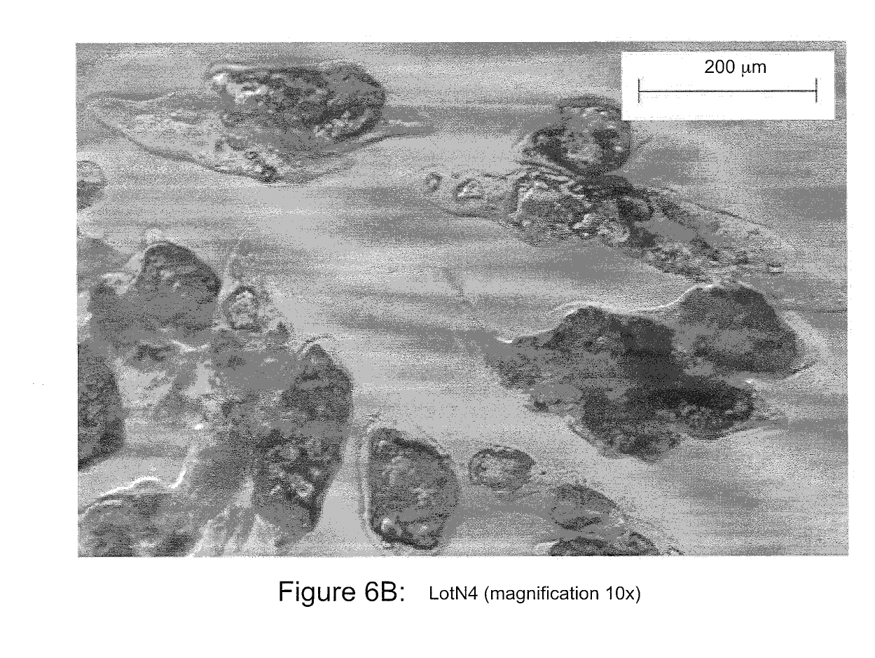

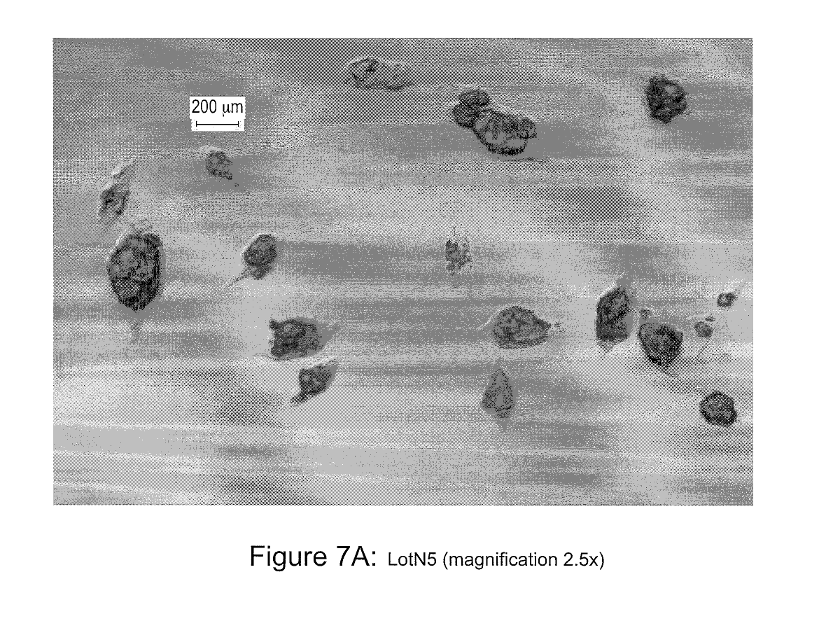

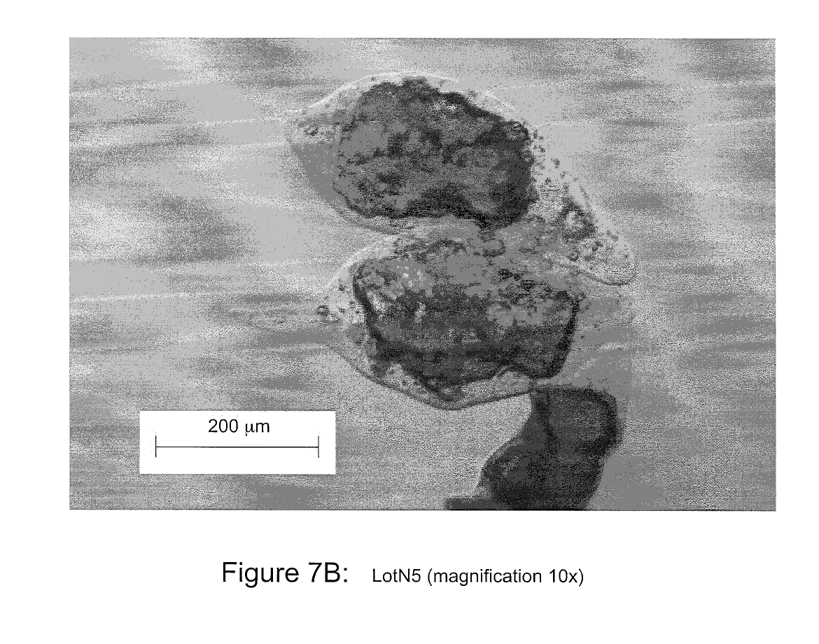

The appearance, particle size distribution, residual solvent content, dissolution rate, and enzymatic activity of the microgranules are analyzed with the techniques as described in previous Example. Microscopic evaluation of the three lots show appropriate polymer deposition around the pancreatin material(FIGS. 5A-B, 6A-B, 7A-B).

The particle size dimension and distribution is measured by sieving according to the following procedure. An amount of microgranules (25-50 g) is poured in a 100 mL HDPE bottle; 0.2% (w/w) of silicon dioxide is sieved through a 150 μm screen, added to the microgranules and manually blended for 2 minutes; the mixture of microgranules and Syloid 244 is sieved with a digital Octagon apparatus for 10 minutes at an amplitude setting of 7.

Table 8 shows that the particle size of microgranules having a 5% coacervated polymeric weight (LotN3) is less than the ones having a 20% coacervated polymer weight (LotN4, LotN5); no significant particle size differences are evident between LotN4 and LotN5 (same weight, different amounts of polyethylene used in preparation process).

TABLE 8
Particle Size Distribution of Microgranules

| | PSD (Sieves) (%) | | | | |
|---|---|---|---|---|---|
| Lot | 425 μm | 300 μm | 212 μm | 150 μm | <150 μm |
| LotN3 | 2.8 | 30.4 | 27.6 | 22.4 | 16.8 |
| (5% Coacervated | | | | | |
| Polymer) | | | | | |
| LotN4 | 6.0 | 49.8 | 29.4 | 13.0 | 1.8 |
| (20% Coacervated | | | | | |
| Polymer) | | | | | |
| LotN5 | 4.5 | 52.6 | 23.8 | 12.8 | 6.3 |
| (20% coacervated | | | | | |
| polymer) | | | | | |

The dissolution rate is determined for LotN3 and LotN4, treated with a 1% solution of sodium docusate in cyclohexane to obtain particles suitable for dissolution rate analysis. The treatment is carried out as follows: a 1% solution of sodium docusate in cyclohexane is prepared by dissolving 6 g of surfactant in 594 g of cyclohexane at room temperature; 22 g of microgranules are poured into a 250 mL beaker containing 100 g of the sodium docusate solution, and then stirred for 15 minutes with a stainless steel propeller (250 RPM). The microgranules are filtered and dried for about 6 h in a fume hood, sieved through a 500 μm opening sieve, and stored in a HDPE container. This method is applied to all subsequent examples. The dissolution rates and enzymatic titers are provided in Table 9.

TABLE 9
Dissolution Rate and Enzyme Activity

| | Coacervated | Li- | Pro- | DRT pH 6.0 protease | | |
|---|---|---|---|---|---|---|
| | Polymer | pase | tease | 5 min % | 15 min % | 30 min % |
| Lot | (%) | (USP/mg) | | (sd) | (sd) | (sd) |
| LotN3 | 5 | 75 | 294 | 83 (2) | 95 (2) | 96 (1) |
| LotN4 | 20 | n.a. | 255 | 54 (2) | 73 (3) | 82 (2) |

The enzymatic activity of the lipase and protease (measured as in previous Example) in the microgranules are close to the theoretic values, and increasing the coating weight of the applied coacervated polymer decreased the dissolution rate values. The residual cyclohexane level of LotN3 is 31 ppm.

### Example 9

### Preparation of Microgranules from Pancreatin of a Selected Particle Size Range (250 μm-500 μm)

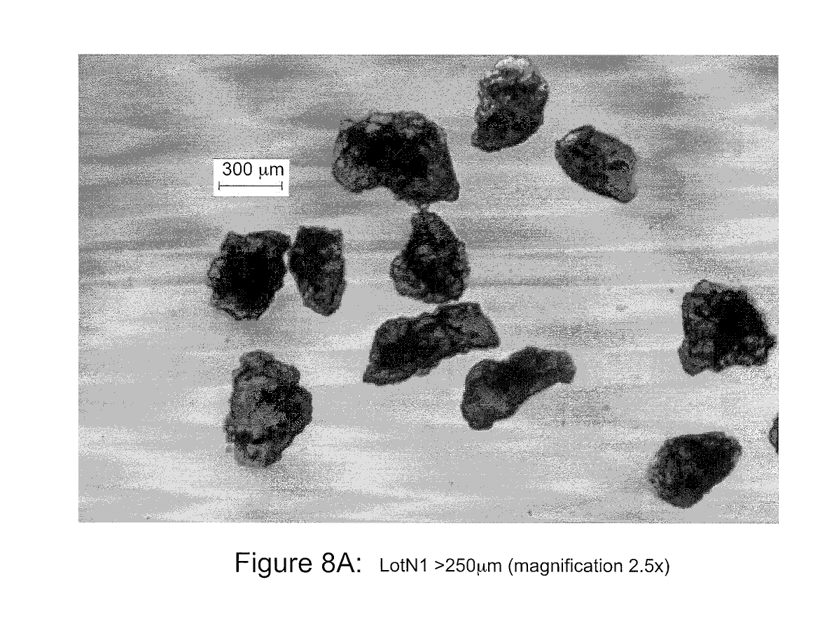

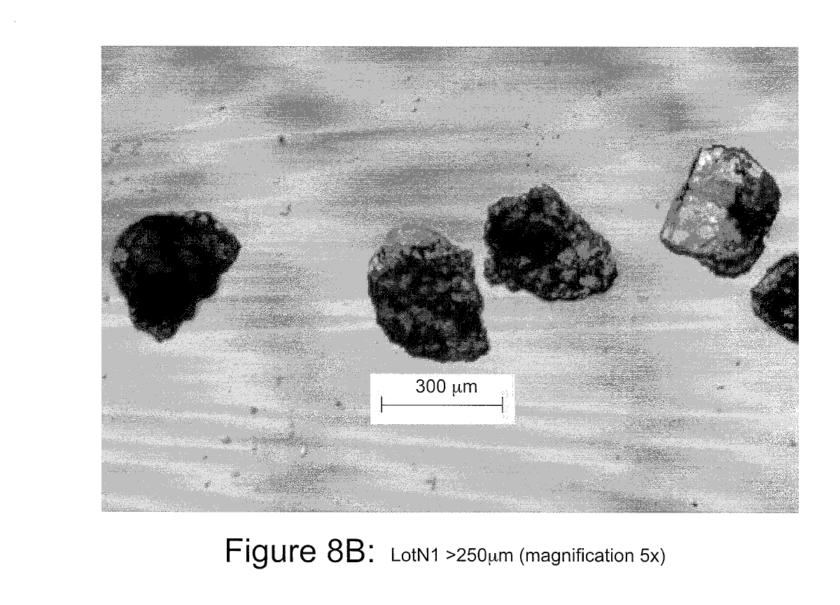

Pancreatin LotN1 is sieved through a 250 μm opening stainless steel sieve, and the small particles (“fines”) are removed. The fraction remaining on the sieve (about 40%) is used to prepare two batches of microgranules. As shown in FIG. 8A-B, the selected material comprises of irregular particles of about 300 μm-400 μm, and very small particles of about 10 μm-30 μm.

Two batches of pancreatin microgranules) are prepared in a laboratory-scale reactor containing 1 kg of cyclohexane, using the process conditions described above with amounts of polyethylene ranging from 0.55 to 1% (Table 10).

TABLE 10
Coacervated polymer Weight, Ethylcellulose Conc., Polyethylene Conc.,
and No. of Washes

| | Coacervated | | | |
|---|---|---|---|---|
| | Polymer | Ethylcellulose | Polyethylene | |
| Lot | Weight (%) | Concentration | Concentration | No. Washes |
| LotN6 | 5 | 0.9% | 0.5% | None |
| LotN7 | 5 | 0.9% | 1.0% | 1 |

The microgranules are filtered and dried overnight (approximately 16 h) in a fume hood, sieved through a 500 μm opening sieve and then stored in a HDPE container. The appearance, residual solvent content, dissolution rate, and enzymatic activity of the product are analyzed.

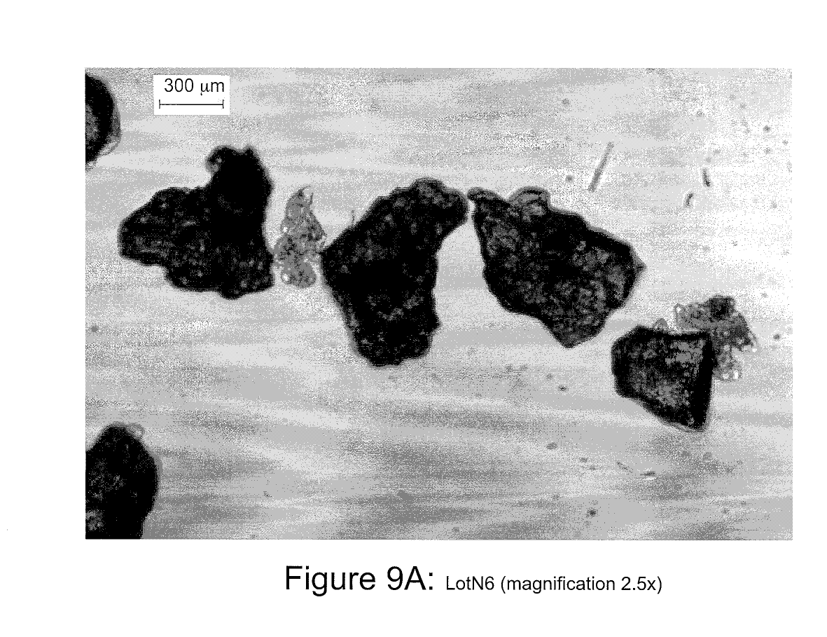

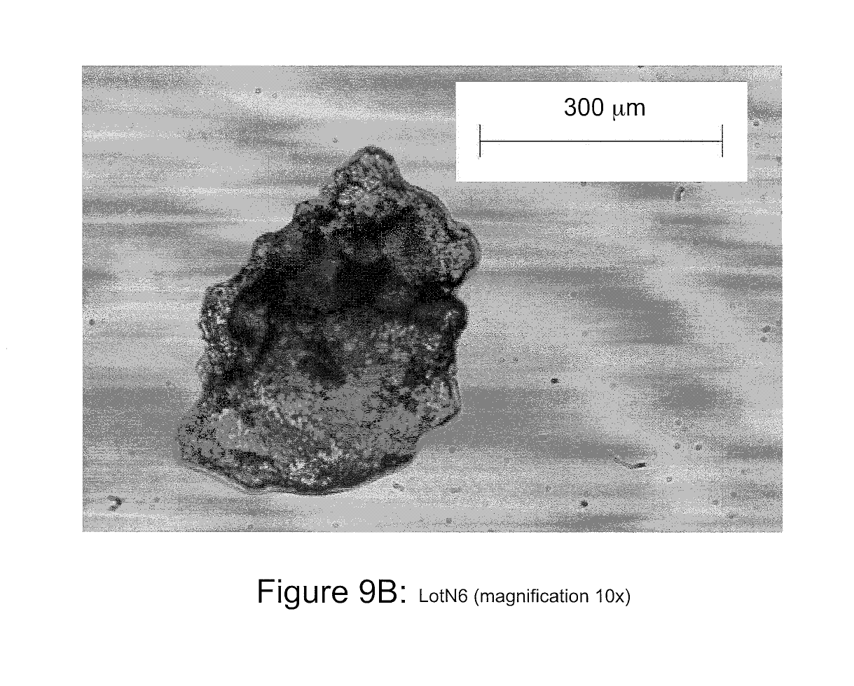

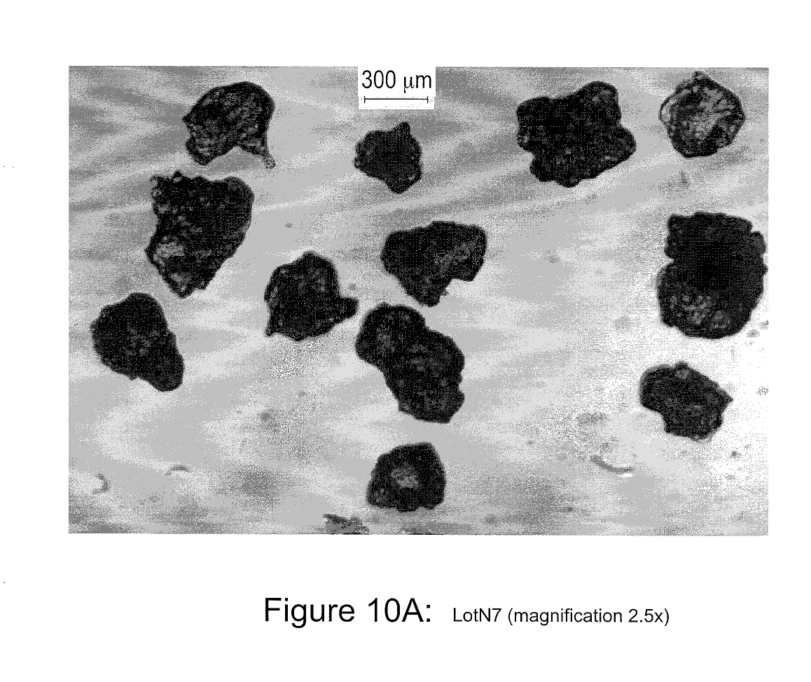

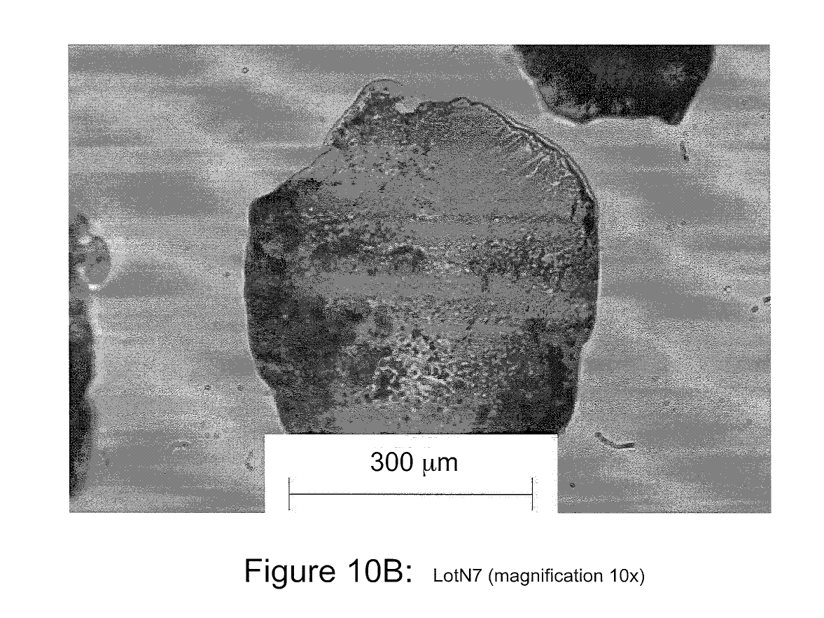

Photomicrographs (FIG. 9A-B and FIG. 10A-B) show that LotN6 (prepared with lower amount of polyethylene) comprises two different populations of microgranules: (a) small microgranules containing several fine particles and (b) microgranules containing the bigger particles (about 300 μm); LotN7 microgranules is more homogeneous and particle size distributions (sieves) of the two batches are similar (Table 11). As expected from the microscope observations, LotN7 has a reduced amount of fine particles.

TABLE 11
Particle Size Distribution of Microgranules

| | PSD (Sieves) (%) | | | | |
|---|---|---|---|---|---|
| Lot | 425 μm | 300 μm | 212 μm | 150 μm | <150 μm |
| LotN6 | 4.3 | 72.4 | 22.7 | 0.6 | 0.0 |
| (5% Coacervated | | | | | |
| Polymer) | | | | | |
| LotN7 | 6.8 | 79.3 | 13.9 | 0.0 | 0.0 |
| (5% Coacervated | | | | | |
| Polymer) | | | | | |

The dissolution rate values of LotN7 (determined after pretreatment with sodium docusate, Table 11) are lower than the corresponding sample obtained from non selected pancreatin (LotN3).

TABLE 12
Dissolution Rate and Assay of Microgranules LotN7.

| | Coacervated | | Protease | | |
|---|---|---|---|---|---|
| | Polymer | Protease | Dissolution Rate at pH 6.0 | | |
| | Weight | Activity | 5 min % | 15 min % | 30 min % |
| Lot. | (%) | (USP/mg) | (sd) | (sd) | (sd) |
| LotN7 | 5 | 303 | 62(3) | 75 (2) | 76 (1) |

### Example 10

### Preparation of Microgranules from Pancreatin of a Selected Particle Size Range (<250 μm)

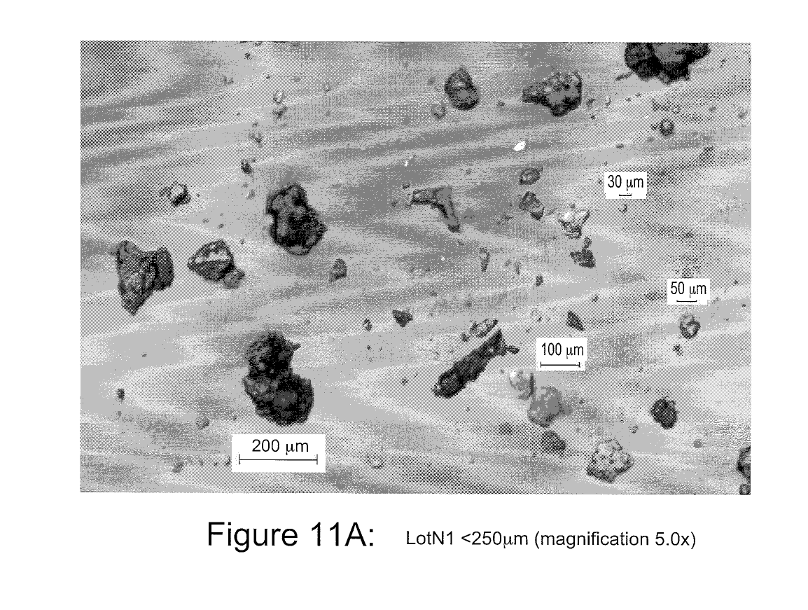

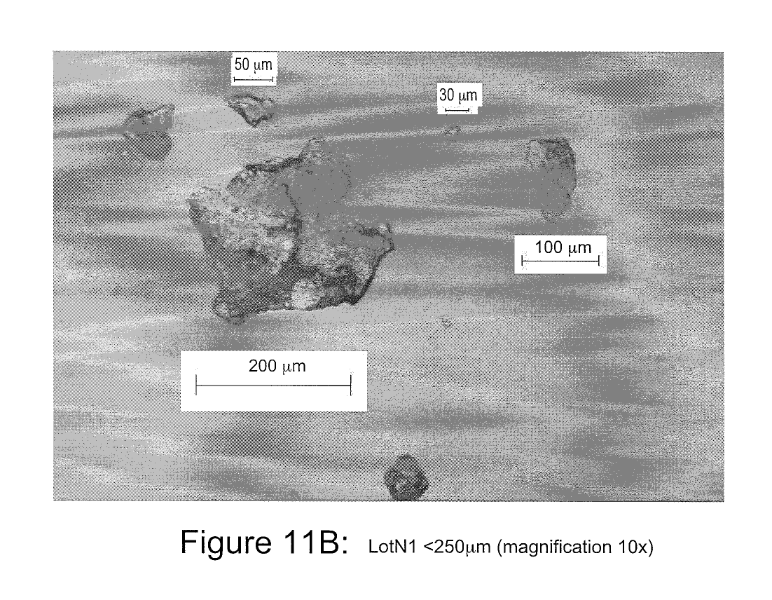

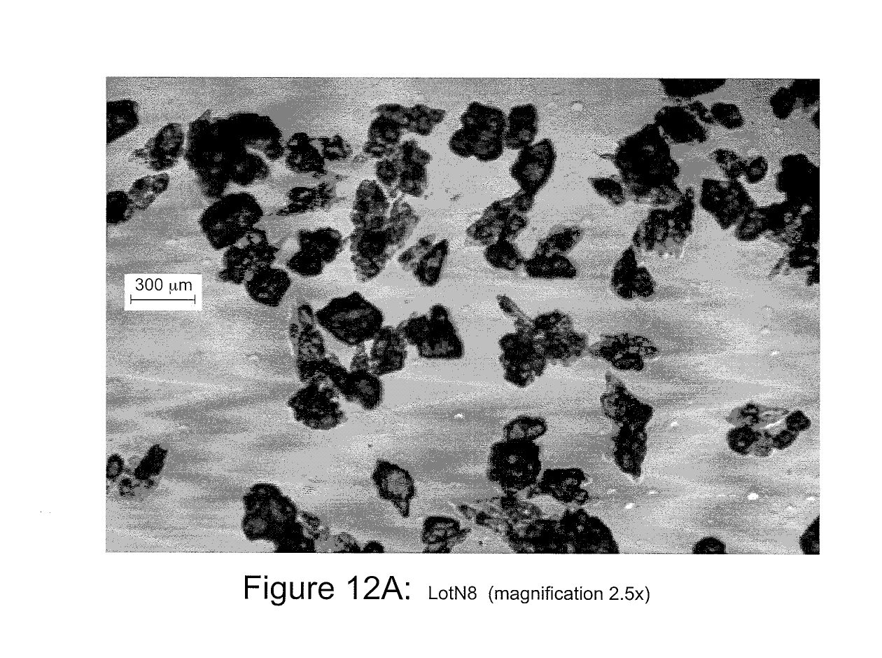

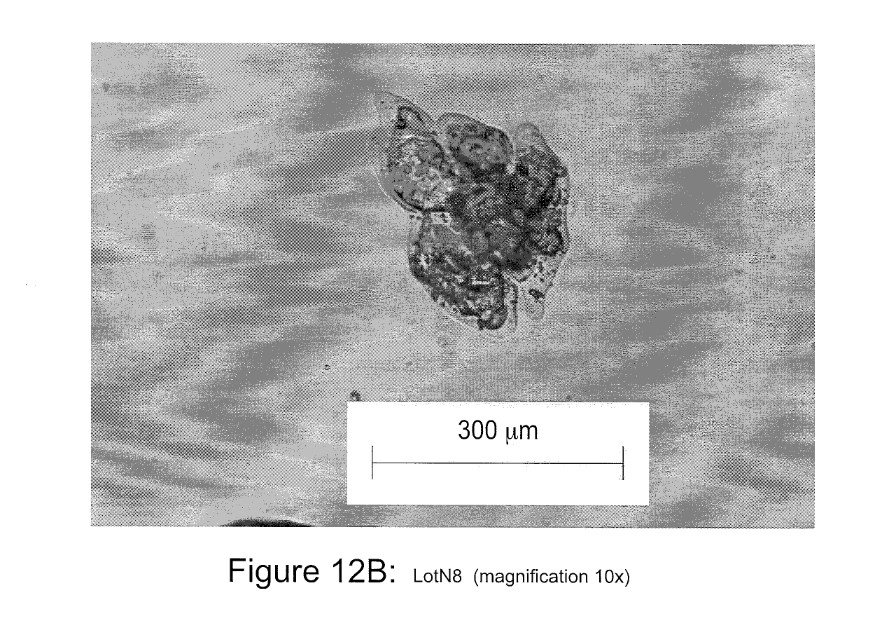

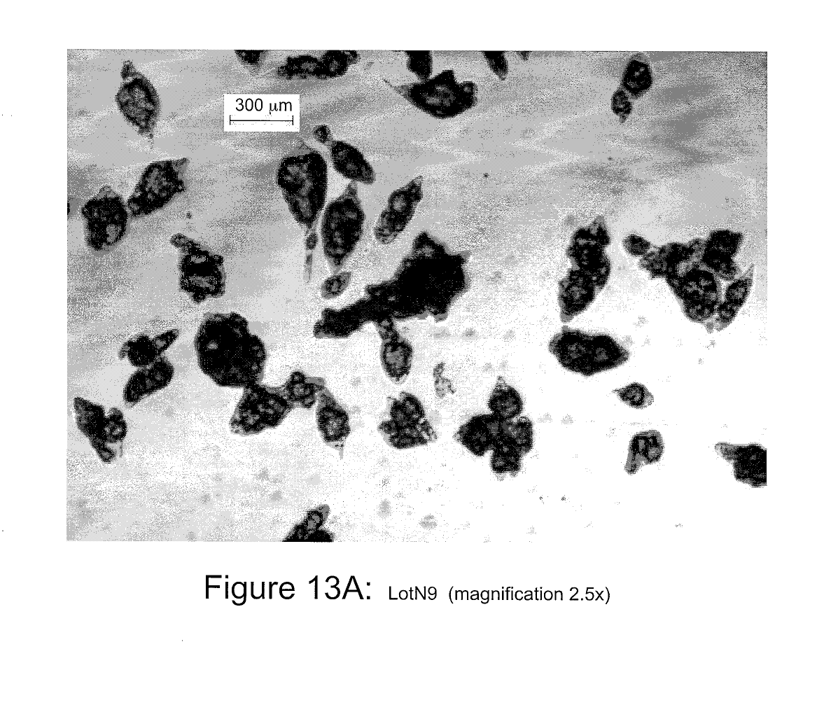

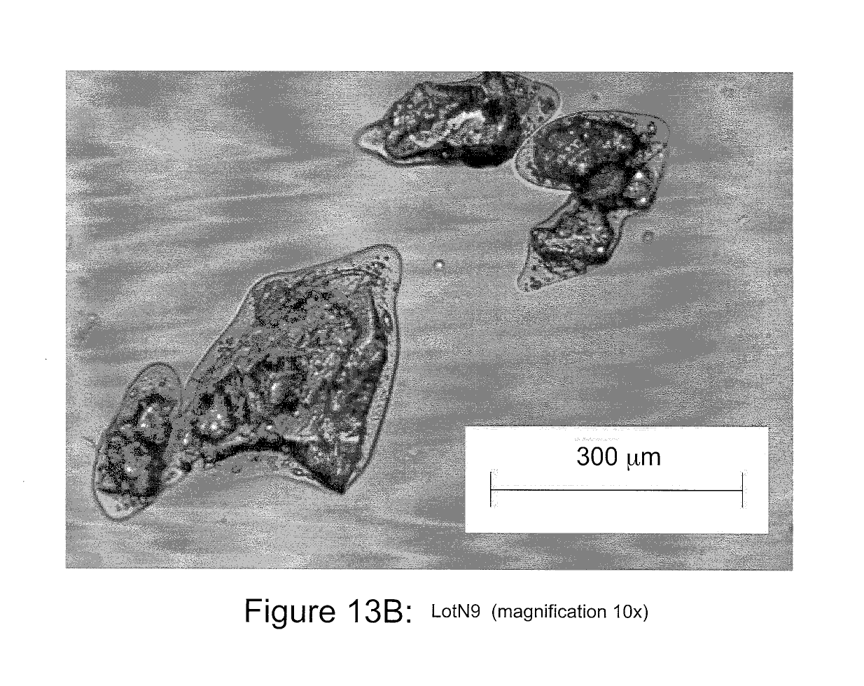

Pancreatin LotN1 is sieved through a 250 μm opening stainless steel sieve and the material that passed through the sieve is used to prepare two batches of microgranules. The selected pancreatin particles range in size from about 20 μm to about 250 μm and are irregularly shaped (FIG. 11A-B).

The selected pancreatin material is introduced in a laboratory-scale reactor containing 1 kg of cyclohexane, using the process conditions described above, and different concentrations of polyethylene. The microgranules are analysed (Table 13).

TABLE 13
Coacervated Ethylcellulose Weight, Ethylcellulose Conc., Polyethylene
Conc., No. Washes

| | Coacervated | | | |
|---|---|---|---|---|
| | Ethylcellulose | Ethylcellulose | Polyethylene | |
| Lot | Weight (%) | Concentration | Concentration | No. Washes |
| LotN8 | 20 | 2% | 0.5% | None |
| LotN9 | 20 | 2% | 2% | 2 |

The microgranules are filtered and dried overnight (approximately 16 h) in a fume hood, sieved through a 500 μm opening sieve and then stored in a HDPE container. The appearance, residual solvent content, dissolution rate and protease activity is and photomicrographs (FIGS. 11A-B and 12A-B) are analyzed with the methods of previous Examples. Microgranules of LotN8 are slightly smaller than those of LotN9 (prepared with higher concentrations of polyethylene).

The sieving method shows that the microgranules of LotN8 are slightly more of the larger microgranules than microgranules of LotN9 (Table 14), which is inconsistent with the microscopic observations. This shows that the different measurement criteria for the microscopic and sieve methods may have an impact on the evaluation and therefore the measurement technique needs to be always specified.

TABLE 14
Particle Size Distribution of Microgranules

| | PSD (Sieve) (%) | | | | |
|---|---|---|---|---|---|
| Lot | 425 μm | 300 μm | 212 μm | 150 μm | <150 μm |
| LotN8 | 6.0 | 38.2 | 37.0 | 16.1 | 2.7 |
| (20% Coacervated | | | | | |
| Polymer) | | | | | |
| LotN9 | 6.4 | 27.0 | 41.0 | 20.5 | 5.1 |
| (20% Coacervated | | | | | |
| Polymer) | | | | | |

The dissolution rate values (pre-treatement method with a 1% solution of sodium docusate) for LotN9 are lower than those for LotN8 (Table 15).

TABLE 15
Dissolution Rate and Protease Activity

| | Coacervated | | Protease | | |
|---|---|---|---|---|---|
| | Polymer | Protease | Dissolution Rate at pH 6.0 | | |
| | Weight | Activity | 5 min % | 15 min % | 30 min % |
| Lot | (%) | (USP/mg) | (sd) | (sd) | (sd) |
| LotN8 | 20 | 233 | 59 (3) | 75 (2) | 82 (1) |
| LotN9 | 20 | 231 | 44 (4) | 67 (1) | 73 (1) |

### Example 11

### Preparation of Pancreatin Microgranules of a Selected Particle Size Range (>250 μm)

Several batches of pancreatin microgranules are prepared from pancreatin having a selected particle size range (>250 μm), a coacervated polymer weight of 5%, and which are produced using the same amount of ethylcellulose and polyethylene used in the preparation of LotN7. The pancreatin microgranules are dried under vacuum at room temperature under a nitrogen gas stream, and sized with 500 μm sieves. The residual cyclohexane level is determined to be 500 ppm. The particle size distribution, dissolution rate and enzymatic titers of the batches are then determined as described before.

TABLE 16
Particle Size Distribution of Pancreatin Microgranules

| | Mean PS | PSD (Sieves) (%) | | | | |
|---|---|---|---|---|---|---|
| Lot | (μm) | 1000 μm | 850 μm | 600 μm | 425 μm | <300 μm |
| LotN10 | 292 | 0.0 | 0.0 | 0.2 | 35.0 | 64.8 |

TABLE 17
Dissolution Rate and Assay of Pancreatin Microgranules

| | | Protease Dissolution | | | |
|---|---|---|---|---|---|
| | | Rate at pH 6.0 (%) | | | Activity |
| | | 5 min. | 15 min. | 30 min. | U USP/mg |
| LotN11 | Protease | 75 (6) | 85 (1) | 89 (2) | 264 (theor. 264) |
| | Lipase² | | | | 83 (theor. 89) |
| | Amylase³ | | | | 376 (theor. 397) |

¹Protease activity: tested using conventional method based on the United States Pharmacopoeia, Pancrelipase: Assay for protease activity.
²Lipase activity: tested using conventional method based on the United States Pharmacopoeia, Pancrelipase: Assay for lipase activity.
³Amylase activity: tested using conventional method based on the United States Pharmacopoeia, Pancrelipase: Assay for amylase activity.

### Example 12

### Preparation of Enteric Coated Pancreatin Microgranules

Pancreatin microgranules LotN10 and LotN11 (Example 11) are loaded in a Glatt-GPCGI fluid bed coater (equipped with a 4″ Wurster insert and a Munters ML 1350 dehumidifier) and sprayed with a coating suspension (HP55:TEC:Talc=10:1:5) having the following composition (% w/w).

TABLE 18
Composition of the Fluid Bed cCoating Suspension

| | |
|---|---|
| Hydroxypropylmethyl cellulose phthalate (HP55) | 7.644% |
| Triethyl citrate (TEC) | 0.764% |
| Talc < 75 μm | 3.822% |
| Acetone | 87.770% |
| Total solid content | 12.23% |

Three batches of enteric coated microgranules are prepared having different coating levels, as shown in Table 19.

TABLE 19
Enteric Coating Levels of Pancreatin Microgranules

| | Enteric Coating | | | Spraying |
|---|---|---|---|---|
| Lot | Weight (%) | Load (g) | Yield | Time (min) |
| LotN12 | 10 | 600 | 97.2% | 102 |
| LotN13 | 33 | 638 | 94.2 | 258 |
| LotN14 | 50 | 600 | 94.3 | 238 |

The final products arc sieved through a 850 μm screen and the enzymatic activity and dissolution rate are determined as shown in Tables 20-21.

TABLE 20
Particle Size Distribution of Enteric Coated Microgranules

| | Mean PSD | PSD (Sieve) (%) | | | | |
|---|---|---|---|---|---|---|
| Lot | (μm) | 1000 μm | 850 μm | 600 μm | 425 μm | <300 μm |
| LotN13 | 397 | 0.2 | 4.2 | 16.6 | 75.6 | 3.4 |
| LotN14 | 422 | 0.0 | 4.0 | 30.6 | 65.0 | 0.4 |

TABLE 21
Composition of Enteric Coated Microgranules (Loss on Drying: 1.2%)

| | mg/g theoretical composition |
|---|---|
| Pancreatin | 475,00 |
| Lipase | 94 × 0.475 = 45 UI/mg |
| Protease | 78 × 0.475 = 132 UI/mg |
| Amylase | 418 × 0.475 = 199 UI/mg |
| Ethylcellulose | 25,00 |
| HP 55 | 312,50 |
| TEC | 31,25 |
| Talc | 156,25 |

TABLE 22
Enzymatic Activity and Dissolution Rate of Coated Microgranules

| | | Dissolution rate | | | |
|---|---|---|---|---|---|
| | | pH 6.0 Protease (%) | | | Activity |
| Lot. | | 5 min | 15 min | 30 min | U USP/mg |
| LotN13 | Protease | 24 (9) | 81 (2) | 86 (1) | 143 (theor. 132) |
| | Lipase | | | | 43 (theor. 45) |
| | Amylase | | | | 206 (theor. 199) |

The resistance of coated pancretin microgranules LotN13 is 43% at pH 5—basket at 37° C. for 60 min and 90% at pH 5—basket at 37° C. for 30 min; the resistance at pH 1.2 is 100% after 30 minutes and 71% after 60 minutes.

### Example 13

### Stability Test of Enteric Coated Pancreatin Microgranules

Stability under accelerated stability conditions of LotN14 is evaluated: the microgranules are sealed into polyethylene sachets, and then the sachets are introduced into thermo-sealed NAPE bags. The products are subjected to stability tests at 40° C. and 75% relative humidity; enzymatic activity and dissolution rate are measured after 1 month of storage (Table 23).

TABLE 23
Enzymatic Activity and Dissolution Rate of Coated Pancreatin
Microgranules

| LotN14 | 0 month | 1 month |
|---|---|---|
| Lipase Activity | 43 U.USP/mg | 44 U.USP/mg |
| Loss on drying | 1.2% | 1.2% |
| Resistance (pH: 5, 37° C., 30 min) | 90% | 88% |
| Dissolution rate (protease): 5 min | 24%(9) | 22%(6) |
| Dissolution rate (protease): 15 min | 81%(2) | 84%(3) |
| Dissolution rate (protease): 30 min | 86%(1) | 88%(5) |

### Example 14

### Preparation and Stability of Coated Pancreatin Microgranules

The starting pancrelipase material (LotM1, 100% pancreatin) has particle size of 300-700 μm (performed using the automatic sieve Octagon Digital equipped with suitable sieves, Endecotte type), the calculated mean geometric diameter is 355 μm. The FIG. 14 shows the PSD of these micropellets which are loaded in the fluid bed for coating. The coating suspension has the following composition: HP-55 10%, talc 1%, triethylcitrate 1%, ethanol 80%, acetone 8%. The fluid bed coating is performed with the following process parameters and equipment configuration: Wurster height 2 cm and length 15 cm, plate type B, spray nozzle 0.8 mm, spray rate 3.5 g/min, atomization pressure 1.4 bar, inlet air temperature 50° C., inlet air velocity 2.0 m/sec, drying temperature 50° C., drying time 15 min, The particles are homogeneous round and have a light brown color. Dosage forms, such as sachets and capsules are prepared with these obtained pancrelipase particles. The packaging in Nialene sachet is obtained by filling manually 2 gr of enteric coated particles, sachets are then soldered at 130° C. The dosage form capsules are obtained by filling each hard gelatin capsule with 500 mg of coated particles, the capsules are stored in HDPE bottles. Intrinsic stability data are reported in Tables 24-25.

TABLE 24
Stability of Enteric Coated Particles in Nialene Sachets (LotM2)

| | | 40° C., | 25° C., | 25° C., | 25° C., |
|---|---|---|---|---|---|
| | | 75% RH | 60% RH | 60% RH | 60% RH |
| Test | T = 0 | 1 month | 3 months | 6 months | 12 months |
| Lipase assay | 26.8 | 23.8 | 24.6 | 25.1 | 25.4 |
| (U/USP/mg)¹ | | | | | |
| Gastroresistance | 92 | 82 | 84 | 91 | 87 |
| (%) | | | | | |
| Loss on drying | — | 2.8 | 2.6 | 2.0 | 2.2 |
| (%) | | | | | |

¹Lipase assay according to European Pharmacopoeia; the obtained U/FIP/mg values are converted into U/USP/mg using the 1:14 factor.

TABLE 24
Stability of Enteric Coated Particles in Gelatin Capsules Packaged in
HDPE Bottles (LotM3)

| | | 40° C., | 25° C., | 25° C., | 25° C., |
|---|---|---|---|---|---|
| | | 75% RH | 60% RH | 60% RH | 60% RH |
| Test | T = 0 | 1 month | 3 months | 6 months | 12 months |
| Lipase assay | 26.8 | 21.8 | 23.1 | 22.4 | 20.5 |
| (U/USP/mg)¹ | | | | | |
| Gastroresistance | 92 | 88 | 94 | 93 | 95 |
| (%) | | | | | |
| Loss on drying | — | 2.8 | 2.9 | 2.3 | 2.8 |
| (%) | | | | | |

¹Lipase assay according to European Pharmacopoeia; the obtained U/FIP/mg values are converted into U/USP/mg using the 1:14 factor.

The stability data show that the enteric coated particles when dosaged in Nialene sachets ensure an acceptable behavior. At 25° C. and 60% R.H., after 12 months, the results are quite satisfactory for both dosage forms. The Nialene sachet packaging material seems to ensure a better behavior in terms of enzymes degradation. After 12 months at 25° C., the lipase activity of the coated particles packaged in Nialene sachets remains practically unmodified, while the same preparation filled in gelatin capsules and packaged in HDPE bottle, show a decrease of the enzymatic activity. The stability of the capsule dosage form containing the same pancreatin enteric coated particles is improved when the coated particles are filled into HPMC (moisture content below 3%) capsules instead of the gelatin capsules.

All these examples demonstrate that the pharmaceutical compositions or dosage forms of digestive enzymes comprising high-dose particles with a sufficiently small, but narrow particle size distribution (e.g., d(0.1)-d(0.9) of 400-800 μm as measured by laser diffraction or with particle size of from about 400 μm to about 600 μm or more preferably particle size of about 250 μm to about 500 μm as measured by sieving), which are suitable for oral or enteral administration, exhibiting acceptable gastroresistant and stability properties, can be of significant interest to pediatric, geriatric and other patients who experience difficulty swallowing conventional tablets or capsules due to dysphagia, thereby improving digestion/absorption of nutrients through better synchronization of transit between digestive enzyme and food particles and hence patient-compliance. These compositions or dosage forms according to the invention comprise high-dose, small sized particles of stabilized digestive enzymes mixtures.

1. A pharmaceutical composition comprising a plurality of particles comprising at least one digestive enzyme, wherein the particles have low particle size dimensions.

2. The pharmaceutical composition of claim 1, wherein the particles have a volume diameter d(v,0.1) of not less than about 400 μm and a volume diameter d(v,0.9) of no more than about 800 μm.

3. The pharmaceutical composition of claim 1, wherein the particles have particle size of less than about 800 μm.

4. The pharmaceutical composition of claim 1 or 3, wherein the particles have particle size of about 400 μm to about 600 μm.

5. The pharmaceutical composition of claim 1 or 3, wherein the particles have particle size of about 250 μm to about 500 μm.

6. The pharmaceutical composition of claim 1, 2, 3, 4 or 5, wherein said particles have at least one coating and the particles comprise: about 40-98 wt. % of digestive enzyme mixture; about 2-20 wt. % a polymer; optionally about 0-30 wt. % inert core; and optionally one or more pharmaceutically acceptable excipients.

7. The pharmaceutical composition of claim 6, wherein said particles comprise at least about 60% by weight of digestive enzyme mixture.

8. The pharmaceutical composition of claim 6, wherein said particles are micropellets and have at least one coating and they comprise: about 40-98 wt. % of digestive enzyme mixture; about 2-10 wt. % a water soluble polymer; optionally about 0-30 wt. % inert core; and optionally one or more pharmaceutically acceptable excipients.

9. The pharmaceutical composition of claim 8, wherein the polymer is a polymer binder.

10. The pharmaceutical composition of claim 8 or 9, wherein the polymer binder is selected from the group consisting of hydroxypropylcellulose, povidone, methylcellulose, hydroxypropyl methylcellulose, carboxyalkylcellulose, polyethylene oxide, vinylpyrrolidone-vinyl acetate copolymer, polysaccharide, and mixtures thereof.

11. The pharmaceutical composition of claim 6, wherein said particles are microgranules and have at least one coating and they comprise: about 80-95 wt. % of digestive enzyme mixture; about 5-20 wt. % a water insoluble polymer; and optionally one or more pharmaceutically acceptable excipients.

12. The pharmaceutical composition of claim 11, where the water insoluble polymer is a coacervated polymer.

13. The pharmaceutical composition of claim 11 or 12, wherein the polymer is ethylcellulose.

14. The pharmaceutical composition of claim 6, 7, 8, 9 or 10, wherein the coating comprises: one or more hydrophilic polymer, one or more plasticizers, and an optional pharmaceutically acceptable hydrophobic material.

15. The pharmaceutical composition of claim 6, 7, 8, 9, 10, 11, 12 or 13, wherein the coating comprises: one or more enteric polymers, and one or more plasticizers.

16. The pharmaceutical composition of claim 1, 2, 3, 4, 5, 6, 7, 8, 9, 10, 11, 12, 13, 14 or 15, wherein the at least one digestive enzyme is selected from the group consisting of pancrelipase, lipase, trypsin, chymotrypsin, chymotrypsin B, pancreatopeptidase, carboxypeptidase A, carboxypeptidase B, glycerol ester hydrolase, phospholipase, phospholipase A2, sterol ester hydrolase, ribonuclease, deoxyribonuclease, alpha-amylase, papain, chymopapain, bromelain, ficin, beta-amylase, cellulase, beta-galactosidase, and mixtures thereof.

17. The pharmaceutical composition of claim 1, 2, 3, 4, 5, 6, 7, 8, 9, 10, 11, 12, 13, 14 or 15, wherein the at least one digestive enzyme is a mixture that comprises at least one lipase, at least one amylase, and at least one protease.

18. The pharmaceutical composition of claim 1, 2, 3, 4, 5, 6, 7, 8, 9, 10, 11, 12, 13, 14 or 15, wherein the at least one digestive enzyme is derived from animal, bacterial, fungal, plant, recombinant origin or is chemically modified.

19. The pharmaceutical composition of claim 14, wherein the hydrophilic polymer is selected from the group consisting of polyvinylpyrrolidone, polyethylene glycol, hydroxypropyl methylcellulose, hydroxypropyl cellulose, low viscosity ethylcellulose, vinylpyrrolidone-vinyl acetate copolymer, polyvinyl alcohol, and mixtures thereof.

20. The pharmaceutical composition of claim 14, wherein the hydrophobic material selected from the group consisting of talc, colloidal silicon dioxide, and magnesium stearate, calcium stearate, zinc stearate, fatty acids glyceryl behenate, glyceryl palmitostearate, and mixtures thereof.

21. The pharmaceutical composition of claim 15, wherein the enteric polymer is selected from the group consisting of cellulose acetate phthalate, hydroxypropyl methylcellulose phthalate, hydroxypropyl methylcellulose acetate succinate, polyvinyl acetate phthalate, and mixtures thereof.

22. The pharmaceutical composition of claim 15, wherein the plasticizer is selected from the group consisting of triacetin, tributyl citrate, triethyl citrate, acetyl tri-n-butyl citrate, diethyl phthalate, dibutyl sebacate, polyethylene glycol, polypropylene glycol, castor oil, acetylated mono-glyceride, acetylated di-glyceride, cetyl alcohol, and mixtures thereof.

23. The composition of claim 15, wherein said enteric coating further comprises an inorganic agent at a ratio of the enteric polymer to the inorganic agent of from about 4:1 to about 1:25 by weight.

24. The pharmaceutical composition of claim 1, 2, 3, 4, 5, 6, 7, 8, 9, 10, 11, 12, 13, 14, 15, 16, 17, 18, 19, 20, 21, 22 or 23, wherein the composition has a moisture content of about 3% or less as measured by loss on drying.

25. The pharmaceutical composition of claim 1, 2, 3, 4, 5, 6, 7, 8, 9, 10, 11, 12, 13, 14 or 15, 16, 17, 18, 19, 20, 21, 22, 23 or 24, wherein the digestive enzyme exhibits a loss of digestive enzyme activity of no more than about 20% after six months of accelerated stability testing.

26. The pharmaceutical composition of claim 25, wherein the accelerated stability testing comprises storing the composition in a sealed Nialene bag at 40° C., 75% relative humidity for 6 months.

27. A method for preparing the pharmaceutical composition of claim 1, 2, 3, 4, 5, 6, 7, 8, 9, 10, 11, 12, 13, 14 or 15, 16, 17, 18, 19, 20, 21, 22 or 23, comprising forming of particles by controlled spheronization, powder layering or coacervation.

28. The method of claim 27, wherein the controlled spheronization is carried out by simultaneously directing a powder mixture comprising at least one digestive enzyme with a volume median diameter d(v,0.5) of no more than about 25 μm and optionally a polymer binder and a flow aid into a powder bed in a product chamber while spraying a polymer binder solution.

29. The method of claim 27, wherein the powder layering is carried out by directing to inert cores suspended in the product chamber a powder mixture comprising at least one digestive enzyme with a volume median diameter, d(v,0.5) of no more than about 25 μm and optionally a polymer binder and a flow aid while spraying a polymer binder solution.

30. The method of claim 27, wherein the coacervation is carried out with a water insoluble polymer dissolved in a hydrophobic organic solvent in presence of a phase separating agent.

31. The method of claim 30, where the water insoluble polymer is ethylcellulose, the hydrophobic organic solvent is cyclohexane and the phase separating agent is polyethylene.

32. A method of preparing a pharmaceutical composition comprising:
a) forming particles comprising at least one digestive enzyme with a d(v,0.1)-d(v,0.9) particle size in the range of about 400-800 μm,
b) optionally coating the particles of step a) with a coating formulation comprising at least one hydrophilic polymer, and at least one plasticizer; and
c) applying an enteric coating comprising an enteric polymer and optionally a plasticizer to the particles of step a) or to the particles of step b).

33. A method of preparing a pharmaceutical composition comprising:
a) forming particles comprising at least one digestive enzyme with particles size of about 250 μm to about 500 μm;
b) optionally coating the particles of step a) with a coating formulation comprising at least one hydrophilic polymer, and at least one plasticizer; and
c) applying an enteric coating comprising an enteric polymer and optionally a plasticizer to the particles of step a) or to the particles of step b).

34. The method of claim 29 or 30, where step a) is carried out by spheronization, powder layering or coacervation.

35. A dosage form comprising the composition of claim 1, 2, 3, 4, 5, 6, 7, 8, 9, 10, 11, 12, 13, 14 or 15, 16, 17, 18, 19, 20, 21, 22 or 23.

36. The dosage form of claim 35 which is a capsule, sachet, or tablet.

37. The dosage form of claim 36, wherein the capsule comprises hydroxypropylmethylcellulose with moisture content of about 3% or less.

38. A method of treating or preventing a patient with a disorder associated with digestive enzyme deficiency comprising administering the pharmaceutical composition of claim 1, 2, 3, 4, 5, 6, 7, 8, 9, 10, 11, 12, 13, 14 or 15, 16, 17, 18, 19, 20, 21, 22 or 23 to a patient for the treatment of digestive disorders, exocrine pancreatic insufficiency, cystic fibrosis, diabetes type I and/or type II.

39. A method of treating or preventing a patient with a disorder associated with digestive enzyme deficiency comprising administering a composition of claim 1, 2, 3, 4, 5, 6, 7, 8, 9, 10, 11, 12, 13, 14 or 15, 16, 17, 18, 19, 20, 21, 22 or 23 in combination with a medicament which increases GI tract pH, to patients in need thereof.
